(19) United States
(12) Patent Application Publication
(10) Pub. No.: US 2026/0341404 A1
(43) Pub. Date: Oct. 29, 2026

(54) PATHOGENICITY PREDICTION FOR PROTEIN MUTATIONS USING AMINO ACID SCORE DISTRIBUTIONS

(71) Applicant: DeepMind Technologies Limited, London (GB)

(72) Inventors: Ziga Avsec, London (GB); Guido Novati, London (GB); Jun Cheng, London (GB)

(21) Appl. No.: 19/118,247

(22) PCT Filed: Oct. 11, 2023
(86) PCT No.: PCT/EP2023/078227

§ 371 Date: Apr. 3, 2025

Related U.S. Application Data
(60) Provisional application No. 63/415,117, filed on Oct. 11, 2022.
(60) Provisional application No. 63/479,653, filed on Jan. 12, 2023.

(51) Int. Cl. G16B 20/50; G16B 30/10; G16B 35/20; G16B 40/20; G16H 50/20

(52) U.S. Cl. CPC G16B 20/50; G16B 30/10; G16B 35/20; G16B 40/20; G16H 50/20

(57) ABSTRACT

Methods, systems, and apparatus, including computer programs encoded on a computer storage medium, for generating a pathogenicity score characterizing a likelihood that a mutation to a protein is a pathogenic mutation, wherein the mutation modifies an amino acid sequence of the protein by replacing an original amino acid by a substitute amino acid at a mutation position in the amino acid sequence of the protein. In one aspect, a method comprises: generating a network input to a pathogenicity prediction neural network, wherein the network input comprises a multiple sequence alignment (MSA) representation that represents an MSA for the protein; processing the network input using the pathogenicity prediction neural network to generate a score distribution over a set of amino acids; and generating the pathogenicity score using the score distribution over the set of amino acids.

### CROSS-REFERENCE TO RELATED APPLICATION

This application claims priority to U.S. Provisional Applications No. 63/415,117, filed on Oct. 11, 2022, and No. 63/479,653, filed on Jan. 12, 2023. The disclosure of the prior applications is considered part of and is incorporated by reference in the disclosure of this application.

### BACKGROUND

This specification relates to predicting pathogenicity of mutant proteins.

A protein is specified by one or more sequences (“chains”) of amino acids. An amino acid is an organic compound which includes an amino functional group and a carboxyl functional group, as well as a side chain (i.e., group of atoms) that is specific to the amino acid. Protein folding refers to a physical process by which one or more sequences of amino acids fold into a three-dimensional (3-D) configuration. The structure of a protein defines the 3-D configuration of the atoms in the amino acid sequences of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a neural network system implemented as computer programs on one or more computers in one or more locations for predicting pathogenicity of mutant proteins.

As used throughout this specification, the term “protein” can be understood to refer to any biological molecule that is specified by one or more sequences (or “chains”) of amino acids. For example, the term protein can refer to a protein domain, e.g., a portion of an amino acid chain of a protein that can undergo protein folding nearly independently of the rest of the protein. As another example, the term protein can refer to a protein complex, i.e., that includes multiple amino acid chains that jointly fold into a protein structure.

A “mutant” protein can refer to a protein that is encoded by a mutated version of a gene, e.g., a mutated version of a deoxyribonucleic acid (DNA) nucleotide sequence. A mutant protein can have a different amino acid sequence than the “original” protein, i.e., that is encoded by the non-mutated version of the gene. The original protein, may be any reference protein i.e. a protein selected as a reference; for example it may be, but need not be, defined by a wild-type version of the protein.

A gene can be mutated in any of a variety of possible ways. For instance, a gene can include a “missense mutation.” A missense mutation can refer to a point mutation in which a single DNA nucleotide change results in a codon that codes for a different amino acid.

A mutant protein can have different properties than the original (non-mutated) protein as a result of having a different amino acid sequence than the original protein. For instance, a mutant protein can have a different stability, a different structure, or a different binding interface than the original protein.

In some cases, one or more biological functions of a protein in a living organism (e.g., a human) rely on the properties of the protein, e.g., the stability, structure, or binding interfaces of the protein. A mutation in the protein can affect the properties of the protein, and by extension, can affect the capacity of the protein to perform its biological functions in the organism. In particular, certain protein mutations can be “pathogenic,” e.g., causing a higher likelihood of disease, e.g., genetic disease, e.g., sickle cell anemia, cystic fibrosis, cancer, etc.

Unless otherwise specified, a “set of amino acids” can refer to the standard twenty (20) amino acids, e.g.: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or any appropriate subset or superset of the standard twenty (20) amino acids, e.g. the proteinogenic amino acids.

A “score distribution” over a set of amino acids, or an “amino acid score distribution,” can refer to data that defines a respective score for each amino acid in the set of amino acids.

A “multiple sequence alignment” (MSA) for an amino acid chain in a protein specifies a sequence alignment of the amino acid chain with multiple additional amino acid chains, e.g., from other proteins, e.g., homologous proteins. More specifically, the MSA can define a correspondence between the positions in the amino acid chain and corresponding positions in multiple additional amino acid chains. A MSA for an amino acid chain can be generated, e.g., by processing a database of amino acid chains using any appropriate computational sequence alignment technique, e.g., progressive alignment construction. The amino acid chains in the MSA can be understood as having an evolutionary relationship, e.g., where each amino acid chain in the MSA may share a common ancestor. The correlations between the amino acids in the amino acid chains in a MSA for an amino acid chain can encode information that is relevant to predicting the structure of the amino acid chain.

An “embedding” of an entity (e.g., a pair of amino acids) can refer to a representation of the entity as an ordered collection of numerical values, e.g., a vector or matrix of numerical values.

The structure of a protein can be defined by a set of structure parameters. A set of structure parameters defining the structure of a protein can be represented as an ordered collection of numerical values. A few examples of possible structure parameters for defining the structure of a protein are described in more detail next.

In one example, the structure parameters defining the structure of a protein include: (i) location parameters, and (ii) rotation parameters, for each amino acid in the protein.

The location parameters for an amino acid can specify a predicted 3-D spatial location of a specified atom in the amino acid in the structure of the protein. The specified atom can be the alpha carbon atom in the amino acid, i.e., the carbon atom in the amino acid to which the amino functional group, the carboxyl functional group, and the side chain are bonded. The location parameters for an amino acid can be represented in any appropriate coordinate system, e.g., a three-dimensional [x, y, z] Cartesian coordinate system.

The rotation parameters for an amino acid can specify the predicted “orientation” of the amino acid in the structure of the protein. More specifically, the rotation parameters can specify a 3-D spatial rotation operation that, if applied to the coordinate system of the location parameters, causes the three “main chain” atoms in the amino acid to assume fixed positions relative to the rotated coordinate system. The three main chain atoms in the amino acid can refer to the linked series of nitrogen, alpha carbon, and carbonyl carbon atoms in the amino acid. The rotation parameters for an amino acid can be represented, e.g., as an orthonormal 3×3 matrix with determinant equal to 1.

Generally, the location and rotation parameters for an amino acid define an egocentric reference frame for the amino acid. In this reference frame, the side chain for each amino acid may start at the origin, and the first bond along the side chain (i.e., the alpha carbon-beta carbon bond) may be along a defined direction.

In another example, the structure parameters defining the structure of a protein can include a “distance map” that characterizes a respective estimated distance (e.g., measured in angstroms) between each pair of amino acids in the protein. A distance map can characterize the estimated distance between a pair of amino acids, e.g., by a probability distribution over a set of possible distances between the pair of amino acids.

In another example, the structure parameters defining the structure of a protein can define a three-dimensional (3D) spatial location of each atom in each amino acid in the structure of the protein.

According to a first aspect there is provided a method performed by one or more computers, the method comprising: generating a pathogenicity score characterizing a likelihood that a mutation to a protein is a pathogenic mutation, wherein the mutation modifies an amino acid sequence of the protein by replacing an original amino acid by a substitute amino acid at a mutation position in the amino acid sequence of the protein, wherein generating the pathogenicity score comprises: generating a network input to a pathogenicity prediction neural network, wherein the network input comprises a multiple sequence alignment (MSA) representation that represents an MSA for the protein; processing the network input using the pathogenicity prediction neural network to generate a score distribution over a set of amino acids; and generating the pathogenicity score based on a difference between: (i) a score, under the score distribution, for the original amino acid, and (ii) a score, under the score distribution, for the substitute amino acid.

In some implementations, the MSA representation comprises a respective embedding corresponding to each position in an amino acid sequence of each protein in the MSA; and generating the MSA representation comprises: masking an embedding in the MSA representation that corresponds to the mutation position in the amino acid sequence of the protein.

In some implementations, processing the network input using the pathogenicity prediction neural network to generate the score distribution over the set of amino acids comprises: processing the MSA representation using an embedding subnetwork of the pathogenicity prediction neural network to generate an updated MSA representation, wherein the updated MSA representation comprises a respective updated embedding corresponding to each position in the amino acid sequence of each protein in the MSA; and processing the updated embedding corresponding to the mutation position in the amino acid sequence of the protein using a projection subnetwork of the pathogenicity prediction neural network to generate the score distribution over the set of amino acids.

In some implementations, the embedding subnetwork of the pathogenicity prediction neural network comprises one or more self-attention neural network layers.

In some implementations, the embedding subnetwork of the pathogenicity prediction neural network comprises one or more row-wise or column-wise self-attention neural network layers.

In some implementations, the pathogenicity prediction neural network has been trained by operations comprising: training the pathogenicity prediction neural network to perform a protein unmasking task; and training the pathogenicity prediction neural network to perform a pathogenicity prediction task.

In some implementations, training the pathogenicity prediction neural network to perform the protein unmasking task comprises, for each of a plurality of training proteins: generating a network input to the pathogenicity prediction neural network based on the training protein, wherein: the network input comprises a MSA representation representing an MSA for the training protein; and the MSA representation comprises a respective masked embedding corresponding to each of one or more masked positions in an amino acid sequence of the training protein; processing the network input to the pathogenicity prediction neural network to generate a network output that, for each masked position in the amino acid sequence of the training protein, defines a respective prediction for an identity of an amino acid located at the masked position in the amino acid sequence of the training protein; and backpropagating gradients of a masking loss through the pathogenicity prediction neural network, wherein the masking loss measures an accuracy of the respective prediction generated by the pathogenicity prediction neural network for each masked position.

In some implementations, the method further comprises training the pathogenicity prediction neural network to perform a protein structure prediction task.

In some implementations, training the pathogenicity prediction neural network to perform the protein structure prediction task comprises, for each of a plurality of training proteins: generating a network input to the pathogenicity prediction neural network based on the training protein, wherein the network input comprises a MSA representation that represents a MSA for the training protein; processing the network input using the pathogenicity prediction neural network to generate an intermediate output of pathogenicity prediction neural network; processing the intermediate output of the pathogenicity prediction neural network using a folding neural network to generate a set of structure parameters defining a predicted structure of the training protein; and backpropagating gradients of a structure loss through the folding neural network and into the pathogenicity prediction neural network, wherein the structure loss measures an error in the predicted structure of the training protein.

In some implementations, the pathogenicity prediction neural network is pre-trained to perform the protein unmasking task and the protein structure prediction task prior to being trained to perform the pathogenicity prediction task.

In some implementations, the pathogenicity prediction neural network has been trained over at least two stages of training comprising a first training stage and a second training stage; wherein the first training stage comprises: training the pathogenicity prediction neural network on a set of pathogenicity training examples, wherein each pathogenicity training example defines: (i) an amino acid sequence of a training protein, (ii) one or more mutations to the amino acid sequence of the training protein, and (iii) a respective target pathogenicity score for each of the one or more mutations; determining a respective filtering score for each training example; and filtering the set of pathogenicity training examples using the filtering scores.

In some implementations, for each training example, determining the filtering score for the training example comprises: determining, for each mutation included in the training example, a predicted pathogenicity score for the mutation using the pathogenicity prediction neural network; and determining, for each mutation included in the training example, an error score for the mutation based on an error between: (i) the predicted pathogenicity score for the mutation, and (ii) the target pathogenicity score for the mutation; and determining the filtering score for the training example based at least in part on the error scores for the mutations included in the training example.

In some implementations, filtering the set of pathogenicity training examples using the filtering scores comprises: determining a probability distribution over the set of pathogenicity training examples based on the filtering scores; sampling a subset of the pathogenicity training examples from the set of pathogenicity training examples in accordance with the probability distribution; and removing the sampled subset of pathogenicity training examples from the set of pathogenicity training examples.

In some implementations, the second training stage comprises: re-training the pathogenicity prediction neural network on the filtered set of pathogenicity training examples.

In some implementations, the method further comprises: determining that the pathogenicity score for the mutation satisfies a threshold; and in response to determining that the pathogenicity score for the mutation satisfies the threshold: classifying the mutation as being a pathogenic mutation.

In some implementations, determining that the pathogenicity score for the mutation satisfies the threshold comprises: determining that the pathogenicity score for the mutation exceeds the threshold.

In some implementations, the method further comprises using the pathogenicity score to identify a cause of a disease in a subject.

In some implementations, the methods described herein are used for obtaining a mutated protein for pest control, e.g., comprising: determining the pathogenicity score for a plurality of mutations of a reference protein each mutation defining a respective mutated protein; and selecting one of the mutations to define the mutated protein for pest control, based on the determined pathogenicity scores.

In some implementations, the methods described herein are used for screening one or more living organisms for the presence of a protein that has a pathogenic mutation, e.g., comprising: obtaining the amino acid sequence of the protein for each of the organisms; for each of the obtained amino acid sequences that includes a mutation, determining the pathogenicity score for the mutation; and processing each determined pathogenicity score to determine whether the mutation is pathogenic.

In some implementations, the methods described herein are used for determining a degree of pathogenicity of a bacterium or virus to a living organism, e.g., comprising: obtaining the amino acid sequence of a manufactured protein, wherein the manufactured protein is made inside the living organism as a result of infection of the living organism by the bacterium or virus, and wherein the manufactured protein is a mutation of a naturally occurring protein in the organism; determining the pathogenicity score of the mutation of the naturally occurring protein; determining a degree of pathogenicity of the bacterium or virus from the pathogenicity score.

In some implementations, the method further comprises providing the pathogenicity score for display on a user interface.

According to another aspect there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect there is provided one or more non-transitory computer storage media storing instructions that when executed by one or more computers cause the one or more computers to perform operations of the methods described herein.

According to another aspect, there is provided a method comprising: maintaining a mutation—pathogenicity database that includes data that defines, for each of a plurality of proteins, a respective pathogenicity score for each of one or more mutations to the protein, wherein the pathogenicity scores have been generated using the methods described herein; obtaining genetic material from a subject; identifying, based on the genetic material from the subject, one or more proteins encoded in the genetic material of the subject that each have one or more mutations; and determining a diagnosis for the subject based on: (i) the mutation-pathogenicity database, and (ii) the protein mutations identified from the genetic material of the subject.

Obtaining genetic material from a subject can include, e.g., obtaining a biological sample from the subject, e.g., a blood sample, or a saliva sample, or a buccal swab, or a tissue biopsy. Genetic material such as deoxyribonucleic acid (DNA) can then be extracted from the biological sample. Genetic testing, e.g., such as DNA sequencing, DNA microarray analysis, polymerase chain reaction (PCR) analysis, or next-generation sequencing (NGS) can be performed to identify one or more proteins encoded in the genetic material of the subject that each have one or more mutations, e.g., as compared to a reference genome.

In some cases, the mutation-pathogenicity database includes data characterizing a plurality of proteins in a human proteome.

In some cases, the mutation-pathogenicity database includes data characterizing all of the proteins in the human proteome.

In some cases, the subject is a human subject.

In some cases, determining the diagnosis for the subject comprises: determining that a particular protein mutation identified from the genetic material of the subject is associated, in the mutation-pathogenicity database, with a pathogenicity score that satisfies a threshold; and in response, diagnosing the subject with a medical condition associated with the particular protein mutation. The threshold can be any appropriate threshold, e.g., 0.5, 0.8, 0.9, or 0.99.

Particular embodiments of the subject matter described in this specification can be implemented so as to realize one or more of the following advantages.

This specification describes a pathogenicity prediction neural network that can generate pathogenicity scores which predict whether protein mutations are pathogenic, e.g., likely to cause disease. To determine whether a mutation at a position in the amino acid sequence of a protein is pathogenic, the pathogenicity prediction neural network can process an MSA for the protein to generate a score distribution over a set of amino acids. The pathogenicity score for the mutation can then be generated based on a difference between: (i) the score, under the score distribution, for the original amino acid at the position, and (ii) the score, under the score distribution, for a substitute amino acid at the position. Processing the MSA enables the pathogenicity prediction neural network to leverage the evolutionary history of the protein which encodes information relevant mutation pathogenicity (as will be described in more detail later in the specification). Moreover, the computation of the pathogenicity score as a difference between amino acid scores assigned to the original and the substitute amino acids encodes the inductive bias that assessing pathogenicity involves comparing the original amino acid to the substitute amino acid.

The pathogenicity prediction neural network can be pre-trained (or jointly trained) to perform an auxiliary task of protein structure prediction. The structure of a protein plays an important role in determining whether mutations to the protein will be pathogenic, and training the pathogenicity prediction neural network to perform the auxiliary protein structure prediction task can enable the pathogenicity prediction neural network to implicitly reason about protein structure. Training the pathogenicity prediction neural network to perform the auxiliary task of protein structure prediction can therefore allow the pathogenicity prediction neural network to be trained on less training data, or over fewer training iterations, or both, thus reducing consumption of computational resources.

The pathogenicity prediction neural network can be pre-trained (or jointly trained) to perform an auxiliary task of protein unmasking. The unmasking task requires the pathogenicity prediction neural network to decode the identities of amino acids at masked positions in an amino acid sequence of a protein based on contextual information from the remaining, unmasked parts of the amino acid sequence. Learning to effectively perform the unmasking task can enable the pathogenicity prediction neural network to implicitly reason about protein biochemistry, and can thereby allow the pathogenicity prediction neural network to be trained on less training data, or over fewer training iterations, or both, thus reducing consumption of computational resources.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example pathogenicity prediction system.

FIG. 2 shows an example architecture of a pathogenicity prediction neural network.

FIG. 3 shows an example training system for training a pathogenicity prediction neural network.

FIG. 4 is a flow diagram of an example process for generating a pathogenicity score characterizing a likelihood that a mutation to a protein is a pathogenic mutation.

FIG. 5 is a flow diagram of an example process for a multi-stage procedure for training a pathogenicity prediction neural network.

FIG. 6A illustrates an example of a MSA representation of an MSA for a protein.

FIG. 6B illustrates an example of a score distribution over a set of amino acids generated by the pathogenicity prediction neural network.

FIG. 7 shows an example protein structure prediction system.

FIG. 8 shows an example architecture of an embedding neural network.

FIG. 9 shows an example architecture of an update block of the embedding neural network.

FIG. 10 shows an example architecture of a MSA update block.

FIG. 11 shows an example architecture of a pair update block.

FIG. 12 shows an example architecture of a folding neural network.

FIG. 13 illustrates the torsion angles between the bonds in the amino acid.

FIG. 14 is an illustration of an unfolded protein and a folded protein.

FIG. 15 is a flow diagram of an example process for predicting the structure of a protein.

FIG. 16 shows an example process for generating a MSA representation for an amino acid chain in a protein.

FIG. 17 shows an example process for generating a respective pair embedding for each pair of amino acids in a protein.

FIG. 18A-B illustrate the performance (e.g., classification accuracy) of the pathogenicity prediction system.

Like reference numbers and designations in the various drawings indicate like elements.

### DETAILED DESCRIPTION

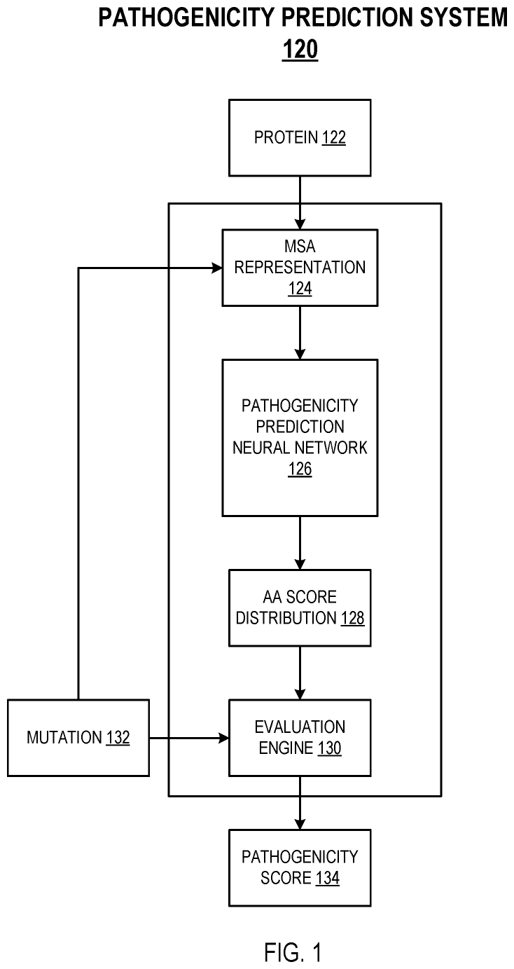

FIG. 1 shows an example pathogenicity prediction system 120. The pathogenicity prediction system 120 is an example of a system implemented as computer programs on one or more computers in one or more locations in which the systems, components, and techniques described below are implemented.

The pathogenicity prediction system 120 is configured to process data defining: (i) an amino acid sequence of a protein 122, and (ii) a mutation 132 to the amino acid sequence of the protein 122, to generate a pathogenicity score 134. The pathogenicity score 134 characterizes a likelihood that the mutation 132 is a pathogenic mutation, e.g., that would cause a higher likelihood of disease in an organism. The amino acid sequence of the protein may be any reference sequence, e.g. a wild-type sequence. The organism may be an animal, e.g. a human, or a plant, or a microorganism such as a bacterium.

The mutation 132 can specify: (i) a mutation position in the amino acid sequence of the protein 122, and (ii) a substitute amino acid for the mutation position in the amino acid sequence (i.e., that, under the mutation, would replace the original amino at the mutation position in the amino acid sequence). The mutation 132 can be a result, e.g., of a missense mutation in a gene coding for the protein 122.

The pathogenicity score 134 can be represented, e.g., as a scalar numerical value, e.g., where higher values of the pathogenicity score can represent a higher likelihood of the protein mutation 122 being pathogenic.

Pathogenicity scores 134 generated by the pathogenicity prediction system 120 for protein mutations can be used in any of a variety of possible applications. For instance, pathogenicity scores 134 can be used for identifying specific genes causing genetic diseases. More specifically, genetic sequencing can be performed for a subject suspected of having a genetic disease, and the genetic data of the subject can be processed to identify a set of mutated protein coding genes in the genome of the subject, e.g. by comparing the gene sequence of a protein with a reference gene sequence. The pathogenicity prediction system 120 described in this specification can be used to generate a respective pathogenicity score the protein mutation caused by each of the mutated protein coding genes. The mutated protein coding genes that are associated with high pathogenicity scores, e.g. scores that are higher than a threshold or that are relatively higher than scores for other proteins, can then be designated as candidate causes of genetic disease in the subject. Some further applications of the system are described later.

The pathogenicity prediction system 120 includes a pathogenicity prediction neural network 126 and an evaluation engine 130, which are each described in more detail next.

To generate a pathogenicity score 134 for a mutation 132, the pathogenicity prediction system 120 obtains data defining a multiple sequence alignment (MSA) for the protein 122, and generates a MSA representation 124 representing the MSA for the protein 122. (Throughout this document, the MSA for a protein should be understood as including the protein itself; that is, the MSA for a protein includes: (i) the protein (a reference version of the protein), and (ii) a set of other proteins that are homologous to the (reference) protein).

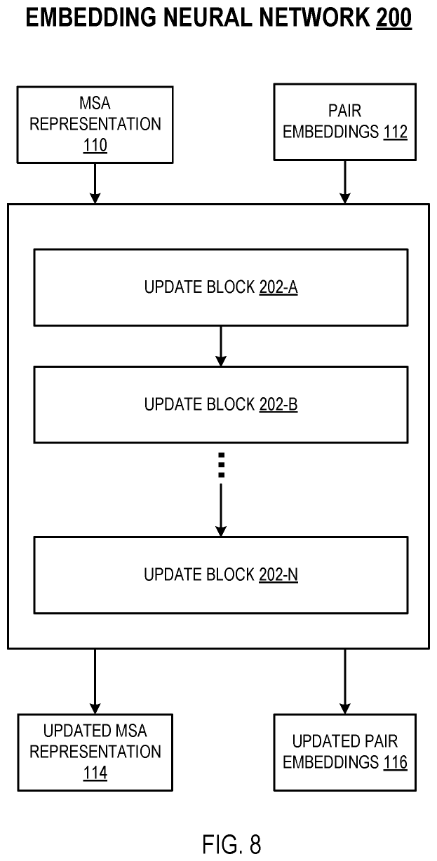

The MSA representation 124 includes a collection of embeddings, including a respective embedding corresponding to each position in the amino acid sequence of each protein included in the MSA. Example techniques for generating an MSA representation 124 for a protein 122 are described in more detail below with reference to FIG. 7.

Optionally, the pathogenicity prediction system 120 can “mask” the embedding in the MSA representation 124 that corresponds to the mutation position in the amino acid sequence of the (reference) protein (i.e., that is specified by the mutation 132). Masking an embedding refers to replacing the embedding by a default (masked) embedding, e.g., a predefined embedding (e.g., having a predefined value, e.g., zero, in each entry) or a random embedding (e.g., where the value of each entry in the embedding is sampled from a probability distribution, e.g., a Gaussian distribution). In general, masking an embedding refers to changing the embedding.

The MSA for a protein encodes information relevant to predicting the pathogenicity of mutations to the protein. For instance, for each position in the amino acid sequence of the protein, the MSA encodes information indicating the frequency with which the amino acid at the position has been altered to a different amino acid over the course of the evolutionary history of the protein. If the amino acid at a position in the amino acid sequence of a protein has mutated frequently over the evolutionary history of the protein, then a mutation at the position may be less likely to be pathogenic. Conversely, if the amino acid at a position in the amino acid sequence of a protein has mutated rarely over the evolutionary history of the protein, then a mutation at the position may be more likely to be pathogenic. Intuitively, if a mutation at a position in the amino acid sequence of a protein causes pathogenicity, then evolutionary pressures may cause amino acid sequences with mutations at the position to appear less frequently in the evolutionary history of the protein.

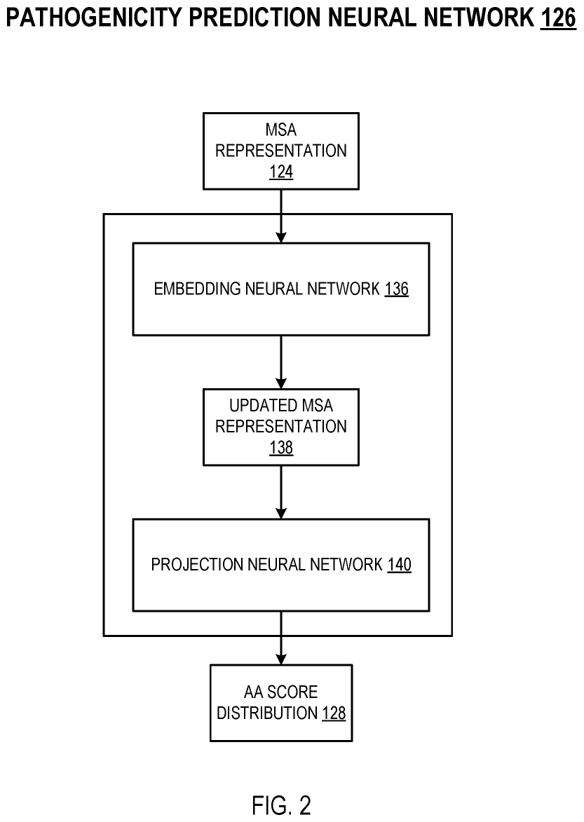

The pathogenicity prediction system 120 processes a network input that includes the MSA representation 124 using the pathogenicity prediction neural network 126 to generate a score distribution 128 over a set of amino acids. An example architecture of the pathogenicity prediction neural network 126 is described in more detail below with reference to FIG. 2.

The evaluation engine 130 generates a pathogenicity score 134 for the mutation 132 at the mutation position based on the amino acid score distribution 128 generated by the pathogenicity prediction neural network 126. For instance, the evaluation engine 130 can generate the pathogenicity score 134 for the mutation 132 based on, e.g. by determining a measure of, a difference between: (i) the score, under (i.e. given by) the score distribution 128, of the original amino acid at the mutation position, and (ii) the score, under the score distribution 128, of the substitute amino acid at the mutation position. The original amino acid at the mutation position refers to the amino acid at the mutation position in the original (i.e., non-mutated) amino acid sequence of the protein 122. That is, the evaluation engine 130 can process an MSA representation with a masked embedding at the mutation position, as previously described, to determine the score distribution 128, and can then generate the pathogenicity score 134 for the mutation 132 from the scores for the original and substitute amino acids in the score distribution, e.g. from a difference between these scores.

In some implementations, the evaluation engine 130 can use the pathogenicity score 134 to classify the mutation 132 as being “pathogenic” or “benign.” For instance, the evaluation engine 130 can classify the mutation 132 as being pathogenic if the pathogenicity score 134 for the mutation 132 satisfies (e.g., exceeds) a threshold. The evaluation engine 130 can classify the mutation 132 as being benign if the pathogenicity score 134 for the mutation does not satisfy (e.g., does not exceed) the threshold. In general the system can be configured so that either a pathogenic mutation is indicated either by a relatively larger score or by a relatively smaller score.

The pathogenicity prediction system 120 can provide the pathogenicity score 134, the classification of the mutation 132, or both, e.g., for storage in a memory, or for presentation to a user by way of a user interface, or for use by a downstream system, or for any other appropriate purpose.

FIG. 2 shows an example architecture of a pathogenicity prediction neural network 126. The pathogenicity prediction neural network 126 can be included in a pathogenicity prediction system, e.g., the pathogenicity prediction system 120 described with reference to FIG. 1.

The pathogenicity prediction neural network 126 is configured to receive a network input that includes an MSA representation 124 of an MSA for a protein. The MSA representation 124 includes a collection of embeddings, including a respective embedding corresponding to each position in the amino acid sequence of each protein included in the MSA. In implementations the embedding corresponding to a “mutation” position in the amino acid sequence of the protein is a masked embedding.

The pathogenicity prediction neural network 126 processes the network input to generate an amino acid score distribution 128. A pathogenicity prediction system 120 can use the amino acid score distribution 128 to generate a pathogenicity score characterizing a likelihood of pathogenicity of a mutation to the mutation position in the amino acid sequence of the protein, as described above with reference to FIG. 1.

The pathogenicity prediction neural network 126 includes an embedding neural network 136 (also termed an embedding subnetwork) and a projection neural network 140 (also termed a projection subnetwork), which are each described in more detail next.

The embedding neural network 136 is configured to process the network input to the pathogenicity prediction neural network 126, including the MSA representation 124, to generate an updated MSA representation 138. The updated MSA representation 138 includes an updated version of each embedding included in the original MSA representation 124, including an updated version of the embedding corresponding to the mutation position in the amino acid sequence of the protein.

The embedding neural network 136 can have any appropriate neural network architecture that enables the embedding neural network 136 to perform its described functions, e.g., processing an MSA representation 124 to generate an updated MSA representation 138. In particular, the embedding neural network 136 can include any appropriate types of neural network layers (e.g., self-attention layers, cross-attention layers, fully connected layers, convolutional layers, etc.) in any appropriate number (e.g., 5 layers, 10 layers, or 15 layers) and connected in any appropriate configuration (e.g., as a linear sequence of layers).

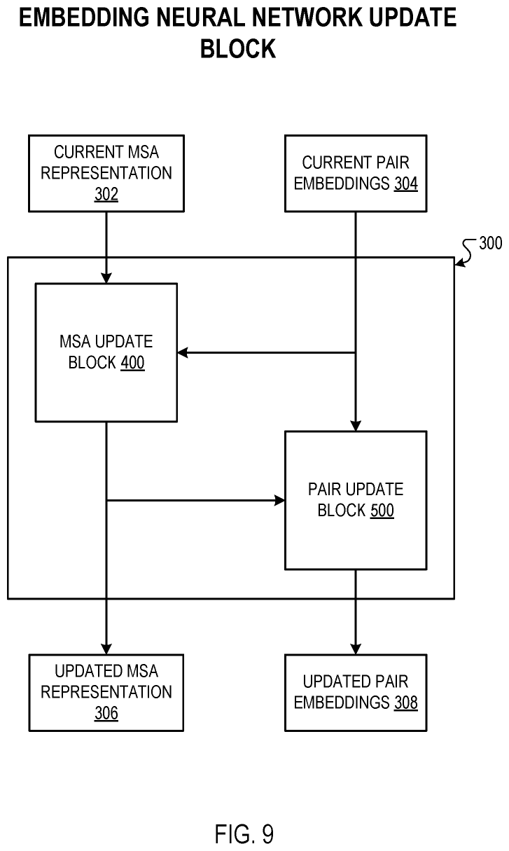

An example implementation of the embedding neural network is described below with reference to FIG. 8. The example embedding neural network described with reference to FIG. 8 is configured to receive an input that additionally includes a set of “pair embeddings” (i.e., in addition to the MSA representation 124), and is configured to generate an output that additionally includes a set of updated pair embeddings (i.e., in addition to the updated MSA representation 138), as described in more detail below with reference to FIG. 8. In general a pair embedding may be an embedding that is derived from a pair of amino acids in an amino acid sequence.

The projection neural network 140 is configured to the process the updated embedding, from the updated MSA representation 138, that corresponds to the mutation position in the amino acid sequence of the protein to generate the score distribution over the set of amino acids.

The projection neural network 140 can have any appropriate neural network architecture that enables the projection neural network 140 to perform its described functions, e.g., processing an embedding from the updated MSA representation to generate an amino acid score distribution. In particular, the projection neural network 140 can include any appropriate types of neural network layer (e.g., a linear layer, self-attention layer, cross-attention layer, fully connected layer, convolutional layer, etc.) in any appropriate number (e.g., 1 layer, 5 layers, 10 layers, or 15 layers) and connected in any appropriate configuration (e.g., as a linear sequence of layers).

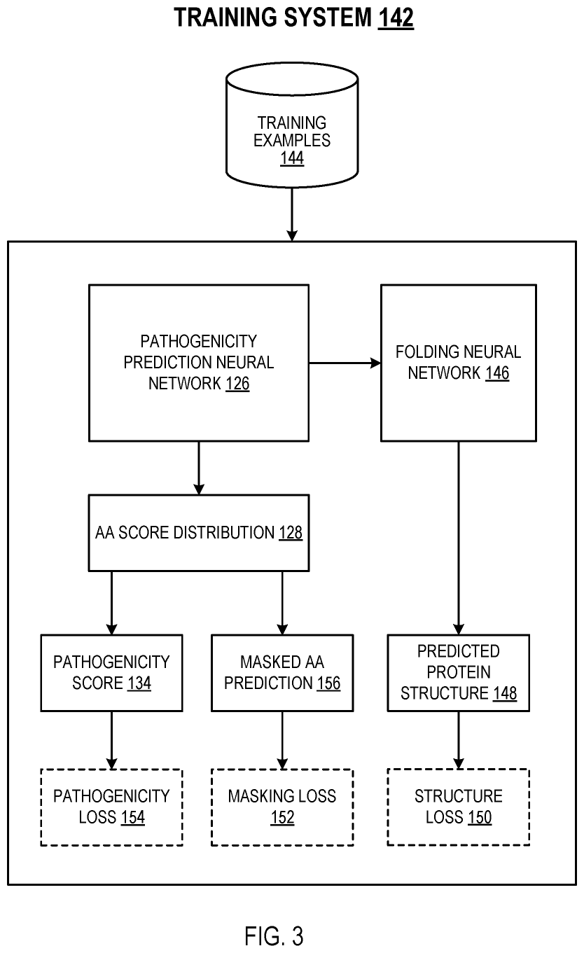

FIG. 3 shows an example training system 142. The training system 142 is an example of a system implemented as computer programs on one or more computers in one or more locations in which the systems, components, and techniques described below are implemented.

The training system 142 is configured to train the pathogenicity prediction neural network 126 on a set of training examples 144. In general the pathogenicity prediction neural network is trained to perform a pathogenicity prediction task; it may also be trained to perform a protein unmasking task (removing an effect of masking an embedding) and or a protein structure prediction task (predicting a structure of a training protein).

Each training example can be, e.g.: a pathogenicity training example, a masked training example, or a structure training example. It can be useful to include masked training examples and structure training examples, but it is not necessary.

A pathogenicity training example defines: (i) an amino acid sequence of a training protein, (ii) one or more mutations to the training protein, and (iii) a respective target pathogenicity score for each of the one or more mutations. Each of the mutations can specify: (i) a mutation position in the amino acid sequence of the training protein, and (ii) a substitute amino acid for the mutation position in the amino acid sequence of the training protein. Some ways of generating the target pathogenicity score are described later. The training proteins may comprise proteins of the organism for which the pathogenicity score is generated, but need not be limited to such proteins.

A masked training example defines: (i) an amino acid sequence of a training protein, and (ii) one or more positions in the amino acid sequence of the training protein that are designated as “masked” positions.

A structure training example defines: (i) an amino acid sequence of a training protein, and (ii) a structure of the training protein (e.g., represented by a set of structure parameters).

The training system 142 can train the pathogenicity prediction neural network 126 on: (i) the pathogenicity training examples to optimize a pathogenicity loss 154, (ii) the masked training examples to optimize a masking loss 152, and (iii) the structure training examples to optimize a structure loss 150, as will be described in more detail next.

To train the pathogenicity prediction neural network 126 on a pathogenicity training example, the training system 142 can provide a network input characterizing the corresponding training protein to the pathogenicity prediction neural network 126. The network input can include a MSA representation of an MSA for the training protein, where the MSA representation includes a respective embedding corresponding to each position in the amino acid sequence of each protein in the MSA. For each mutation specified by the training example, the training system 142 masks the embedding corresponding to the mutation position of the mutation in the amino acid sequence of the training protein.

The training system 142 processes the network input for the training protein using the pathogenicity prediction neural network 126 to generate a respective amino acid score distribution for the mutation position of each mutation specified by the training example. For each mutation specified by the training example, the training system then generates a respective predicted pathogenicity score 134 based on the amino acid score distribution generated by the pathogenicity prediction neural network 126 for the mutation position. (Example techniques for generating a pathogenicity score for a mutation from an amino acid score distribution are described above with reference to FIG. 1).

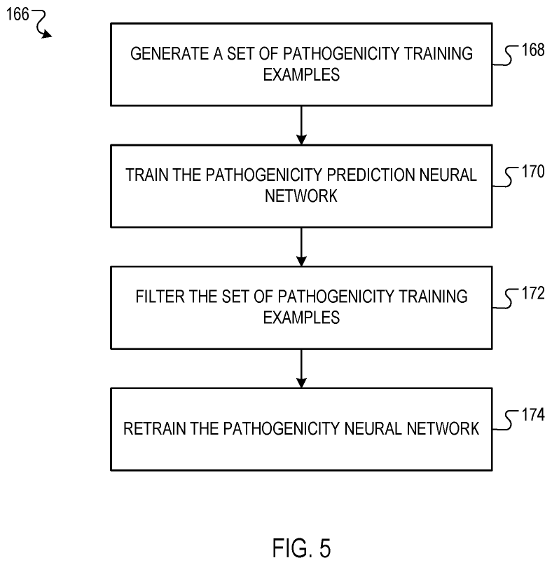

The training system 142 evaluates the pathogenicity loss 154 for each mutation specified by the pathogenicity training example, and then determines the overall pathogenicity loss for the pathogenicity training example as a combination (e.g., an average or sum) of the pathogenicity loss for each mutation. The training system 142 can evaluate the pathogenicity loss for a mutation based on an error (e.g., a hinge loss, or a cross-entropy loss, or any other appropriate loss) between: (i) the predicted pathogenicity score for the mutation, and (ii) the target pathogenicity score for the mutation. The target pathogenicity score for the mutation can be obtained as described later with reference to FIG. 5. As one example, the target pathogenicity score can have a value that indicates a prevalence of the mutation in a population of an organism or group of organisms. Then the organism or group of organisms can include an organism for which the pathogenicity prediction system 120 is used (in inference) to generate a pathogenicity score—but as there can be substantial similarities between different biological organisms it is not essential that the pathogenicity prediction system 120 is trained on data from the same organism for which it is used in inference.

The training system 142 can determines gradients of the pathogenicity loss 154 with respect to the parameters of the pathogenicity prediction neural network 126, e.g., using backpropagation. The training system 142 can then adjust the current values of the parameters of the pathogenicity prediction neural network 126 using the gradients, e.g., using an appropriate gradient descent optimization technique.

To train the pathogenicity prediction neural network 126 on a masked training example, the training system can provide a network input characterizing the corresponding training protein to the pathogenicity prediction neural network 126. The network input can include an MSA representation representing an MSA for the training protein, where the MSA representation includes a respective embedding corresponding to each position in the amino acid sequence of each protein in the MSA. The training system 142 can mask each embedding in the MSA representation 124 that corresponds to a position in the amino acid sequence of the training protein that has been designated by the masked training example as a masked position.

The training system 142 then processes the network input for the training protein using the pathogenicity prediction neural network 126 to generate a respective amino acid score distribution corresponding to each masked position in the amino acid sequence of the training protein. The training system 142 evaluates a masking loss for each masked position, and then determines the overall masking loss for the training example as a combination (e.g., an average or sum) of the masking loss for each masked position. The training system 142 can evaluate the masking loss for a masked position based on the score, under the score distribution for the masked position, for the amino acid at the masked position in the training protein, e.g. based on a change in the score due to the masking. The masking loss can measure an accuracy of the respective prediction generated by the pathogenicity prediction neural network for each masked position.

The training system 142 can determines gradients of masking loss 152 with respect to the parameters of the pathogenicity prediction neural network 126, e.g., using backpropagation. The training system 142 can then adjust the current values of the parameters of the pathogenicity prediction neural network 126 using the gradients, e.g., using an appropriate gradient descent optimization technique.

By training the pathogenicity prediction neural network 126 on the masking loss 152, the training system 142 trains the pathogenicity prediction neural network 126 to perform an unmasking task. The unmasking task requires the pathogenicity prediction neural network to decode the identities of amino acids at masked positions in an amino acid sequence of a protein based on contextual information from the remaining, unmasked parts of the amino acid sequence. Learning to effectively perform the unmasking task can implicitly encode an understanding of protein biochemistry in the parameter values of the pathogenicity prediction neural network, and can thereby enhance the performance of the pathogenicity prediction neural network 126 on the task of pathogenicity prediction.

To train the pathogenicity prediction neural network 126 on a structure training example, the training system 142 can provide a network input characterizing the corresponding training protein to the pathogenicity prediction neural network 126. The network input can include an MSA representation representing an MSA for the training protein. The training system 142 processes the network input for the training protein using the pathogenicity prediction neural network 126, and provides an intermediate output of the pathogenicity prediction neural network to a folding neural network 146. (An intermediate output of a neural network refers to an output generated by one or more hidden layers of the neural network; any layer may be used). The folding neural network 146 is configured to process the intermediate output of the pathogenicity prediction neural network 126 to generate data defining a predicted structure 148 of the training protein.

The folding neural network 146 can be configured to receive any appropriate intermediate output of the pathogenicity prediction neural network 126. For instance, the pathogenicity prediction neural network 126 can include an embedding neural network, as described with reference to FIG. 8, and the folding neural network 146 can be configured to receive some or all of the outputs of the embedding neural network. For an embedding neural network having the architecture described below with reference to FIG. 8, the folding neural network can receive the updated MSA representation and/or the updated pair embeddings generated by the embedding neural network.

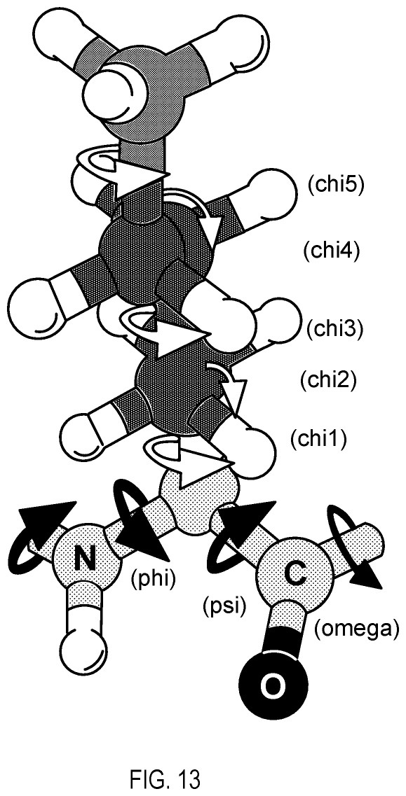

The folding neural network can have any appropriate neural network architecture that enables the folding neural network to perform its described functions, in particular, processing an intermediate output of the pathogenicity prediction neural network to generate a predicted protein structure. In particular, the folding neural network can include any appropriate types of neural network layers (e.g., self-attention layers, cross-attention layers, fully connected layers, convolutional layers, etc.) in any appropriate number (e.g., 5 layers, 10 layers, or 15 layers) and connected in any appropriate configuration (e.g., as a linear sequence of layers). An example implementation of a folding neural network is described below with reference to FIG. 12.

The structure loss 150 for the structure training example measures an error between: (i) the predicted protein structure 148 generated by the folding neural network 146, and the (ii) target protein structure specified by the structure training example. An example of a structure loss is described below, e.g., with reference to equations (2)-(4). The training system 142 determines gradients of the structure loss 150 with respect to the parameters of the pathogenicity prediction neural network 126 and the folding neural network 146, e.g., using backpropagation. The training system 142 can then adjust the current values of the parameters of the pathogenicity prediction neural network 126 and the folding neural network 146 using the gradients, e.g., by an appropriate gradient descent optimization technique. That is, the training system 142 backpropagates gradients of the structure loss 150 through the folding neural network 146 and into the pathogenicity prediction neural network 126.

The folding neural network 146 is not required after the training of the pathogenicity prediction neural network 126. Therefore the folding neural network 146 can be discarded after the training of the pathogenicity prediction neural network 126.

By training the pathogenicity prediction neural network 126 and the folding neural network 146 on the structure loss 150, the training system 142 trains the pathogenicity prediction neural network 126 and the folding neural network 146 to jointly perform the task of protein structure prediction. Learning to effectively perform the protein structure prediction task can implicitly encode an understanding of protein biochemistry in the parameter values of the pathogenicity prediction neural network, and can thereby enhance the performance of the pathogenicity prediction neural network on the task of pathogenicity prediction.

More specifically, the structure of a protein encodes information about the likely pathogenicity of mutations to the protein. For instance, proteins can fold to nest hydrophobic amino acids in the interior of the protein, while exposing hydrophilic amino acids on the exterior of the protein. A mutation to a protein that would place a hydrophobic amino acid on the exterior of the protein could substantially change the properties of the protein, e.g., by causing the protein to become unstable, or by changing the shape of the protein, and thus has the potential to be pathogenic. Training the pathogenicity prediction neural network 126 on the structure loss 150 can enable the pathogenicity prediction neural network 126 to reason about protein structure and therefore perform pathogenicity prediction more accurately.

In some implementations, the training system 142 first trains the pathogenicity prediction neural network 126 to optimize the masking loss 152 and the structure loss 150. The training system 142 then disables the masking loss 152 and the structure loss 150, and trains the pathogenicity prediction neural network 126 solely on the pathogenicity loss 154. That is, the training system 142 can pre-train the pathogenicity prediction neural network 126 on the masking loss 152 and the structure loss 150, and then fine tune the pathogenicity prediction neural network 126 on the pathogenicity loss. In some cases, during the fine tuning, the training system 142 can train the pathogenicity prediction neural network on both the pathogenicity loss and the structure loss 150 (i.e., rather than disabling the structure loss 150).

Pre-training the pathogenicity prediction neural network on the masking loss 152 and the structure loss 150 prior to fine tuning the pathogenicity prediction neural network on the pathogenicity loss 154 can enhance the performance of the pathogenicity prediction neural network on the task of pathogenicity prediction. In particular, the pre-training can generate an effective initialization for the parameter values of the pathogenicity prediction neural network, and can enable the pathogenicity prediction neural network to achieve acceptable performance on the task of pathogenicity prediction using little training data and few training iterations.

In some cases, the pathogenicity training examples used for training the pathogenicity prediction neural network can be “noisy,” e.g., can be generated by a process that results in a non-negligible number of pathogenicity training examples with inaccurate target pathogenicity scores. To address this issue, the training system 142 can filter, i.e. remove, noisy pathogenicity training examples from the training data over the course of training, and thus regularize and improve the training of the pathogenicity prediction neural network. An example of a multi-stage training procedure for training the pathogenicity prediction neural network 126 while filtering noisy pathogenicity training examples from the training data is described in more detail below with reference to FIG. 5.

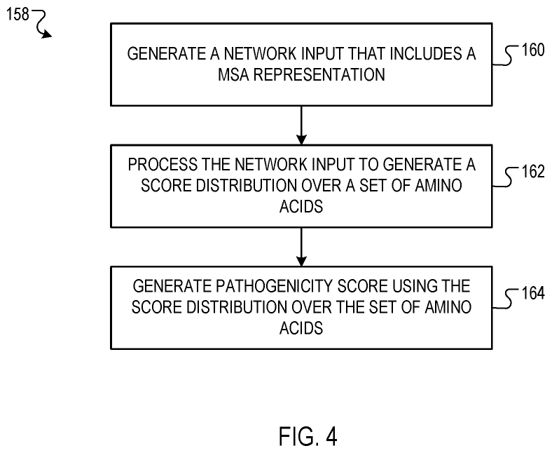

FIG. 4 is a flow diagram of an example process 158 for generating a pathogenicity score characterizing a likelihood that a mutation to a protein is a pathogenic mutation. The mutation modifies an amino acid sequence of the protein by replacing an original amino acid by a substitute amino acid at a mutation position in the amino acid sequence of the protein. In implementations the process involves obtaining data representing the mutation. For convenience, the process 158 will be described as being performed by a system of one or more computers located in one or more locations. For example, a pathogenicity prediction system, e.g., the pathogenicity prediction system 120 of FIG. 1, appropriately programmed in accordance with this specification, can perform the process 158.

The system generates a network input to a pathogenicity prediction neural network (160). The network input includes a multiple sequence alignment (MSA) representation that represents an MSA for the protein. In implementations an embedding in the MSA representation that corresponds to the mutation position in the amino acid sequence of the protein is masked.

The system processes the network input using the (trained) pathogenicity prediction neural network to generate a score distribution over a set of amino acids (162). In implementations the pathogenicity prediction neural network has been trained to optimize (minimize) a pathogenicity loss characterizing a difference between a pathogenicity of a protein mutation and a prediction of the pathogenicity of the protein mutation from the pathogenicity prediction system, i.e. derived from the score distribution over a set of amino acids, more particularly from the pathogenicity score for the protein mutation.

The system generates the pathogenicity score based on a difference between: (i) a score, under the score distribution, for the original amino acid, and (ii) a score, under the score distribution, for the substitute amino acid (164).

FIG. 5 is a flow diagram of an example process 166 for implementing a multi-stage procedure for training a pathogenicity prediction neural network. For convenience, the process 166 will be described as being performed by a system of one or more computers located in one or more locations. For example, a training system, e.g., the training system 142 of FIG. 3, appropriately programmed in accordance with this specification, can perform the process 166.

The system generates a set of pathogenicity training examples (168). Each pathogenicity training example defines: (i) an amino acid sequence of a training protein, (ii) one or more mutations to the training protein, and (iii) a respective target pathogenicity score for each of the one or more mutations.

In some implementations the system can generate or obtain the set of pathogenicity training examples based on a dataset that characterizes frequency of occurrence of protein mutations in a population of organisms, e.g., a population of humans, or a population of primates, or a population of mammals or of other animals, or a population of plants, or a population of microorganisms. Also or instead the system can use known biological data e.g. from an appropriate database, to obtain a measure of pathogenicity of a mutation.

In particular, the system can identify each protein mutation that occurs in at least a threshold percentage of organisms in the population as being a “benign” mutation, i.e., non-pathogenic mutation. Intuitively, a protein mutation that is severely pathogenic is unlikely to appear frequently in the population, e.g., because organisms having a severely pathogenic mutation would likely be more likely to be removed from the population and less likely to reproduce. Thus protein mutations that occur with at least the threshold frequency in the population are less likely to be severely pathogenic, and can be identified as being benign mutations for the purpose of generating training examples for training the pathogenicity prediction neural network.

The system can designate a protein mutation as being a pathogenic mutation if the protein mutation has not occurred in the population of organisms. Thus, to identify pathogenic mutations, the system can randomly generate a set of protein mutations, remove any mutations that have occurred in the population of organisms, and then designate the remaining mutations as being pathogenic mutations. Intuitively, a protein mutation may not occur in the population because: (i) the population (effective population size) is not sufficiently large for the mutation to have been reflected in at least one organism in the population, or (ii) the protein mutation is severely pathogenic such that organisms having the protein mutation are highly unlikely to reproduce or survive.

After identifying a set of benign mutations and a set of pathogenic mutations, as described above, the system can proceed to generate or obtain pathogenicity training examples. In particular, to generate a pathogenicity training example, the system can identify a training protein and one or more protein mutations associated with the training protein. The system can associate a first pathogenicity score, e.g., of zero (0), with each mutation to the training protein that has been designated as being benign, and a second pathogenicity score, e.g., of one (1), with each mutation to the training protein that has been designated as being pathogenic.

It will be appreciated that the above-described procedure for identifying benign and pathogenic protein mutations may result in the generation of “noisy” pathogenicity training examples. For instance, pathogenic protein mutations occur in populations, and therefore the appearance of a protein mutation at least the threshold number of times in the population does not necessarily imply that the protein mutation is benign. Conversely, a protein mutation may fail to occur in the population because the population (effective population size) is not sufficiently large, rather than because the protein mutation is pathogenic. To address this issue, the system can filter the set of pathogenicity training examples to remove pathogenicity training examples that are likely to be inaccurate, as will be described below with reference to step (172).

The system trains the pathogenicity prediction neural network on a set of training examples (170). The set of training examples includes the pathogenicity training examples generated at step (168), as well as masked training examples and structure training examples. Example techniques for training the pathogenicity prediction neural network on a set of training examples including pathogenicity training examples, masked training examples, and structure training examples are described in more detail with reference to FIG. 3.

In some implementations the system filters the set of pathogenicity training examples (172). In particular, for each protein mutation, the system can generate a predicted pathogenicity score for the protein mutation using the pathogenicity prediction neural network. The system then generates an error score for the protein mutation based on an error between: (i) the target pathogenicity score, and (ii) the predicted pathogenicity score, for the protein mutation. Here and elsewhere the target pathogenicity score for a protein mutation can be, e.g., a binary 0/1 score indicating whether the protein mutation was designated as being benign or pathogenic, e.g., at step (168). Intuitively, the error score for a protein mutation being large indicates that the trained pathogenicity prediction neural network generated a (confident) prediction that opposes the original designation of the protein mutation as being benign or pathogenic. A large error score for a protein mutation can indicate that the protein mutation was mislabeled as being benign or pathogenic.

The system can generate a filtering score for each pathogenicity training example at least in part on the error scores for the mutations included in the amino acid sequence of the corresponding training protein. For instance, the system can generate a filtering score for a pathogenicity training example based at least in part on a combination, e.g. product, of the error scores for the mutations included in the amino acid sequence of the corresponding training protein. The system can use the filtering scores to filter the pathogenicity training examples in many ways. As one example the system can generate a probability distribution over the set of pathogenicity training examples using the filtering scores (e.g. with a softmax), sample multiple pathogenicity training examples in accordance with the probability distribution (e.g. so that examples with higher filtering scores are more likely to be selected), and filter (remove) the sampled pathogenicity training examples from the set of pathogenicity training examples.

The filtering score for a pathogenicity training example can additionally be based on other aspects of the corresponding training protein, i.e., in addition to the error scores for the protein mutations. For instance, the filtering score for a pathogenicity training example can have a higher value if the number of (pathogenic or benign) mutations included in the corresponding training protein significantly deviates (e.g., by at least a threshold amount) from an expected number of (pathogenic or benign) mutations, e.g., that are typically observed in proteins. A training protein with a significantly higher or lower number of (pathogenic or benign) mutations than the expected number of (pathogenic or benign) mutations can be easily differentiated from natural proteins by the pathogenicity prediction neural network, which can compromise the effectiveness of training.

Filtering the set of pathogenicity training examples based on the filtering scores can improve the quality of the set of pathogenicity training examples, e.g., by removing training examples with mislabeled protein mutations, and by removing training examples with unnatural artifacts.

The system can retrain the pathogenicity prediction neural network on a set of training examples that includes the filtered set of pathogenicity training examples (174). That is, the filtered (removed) pathogenic training examples are not used during the retraining of the pathogenicity prediction neural network. The retrained pathogenicity prediction neural network can therefore be trained to achieve a higher prediction accuracy over fewer training iterations, e.g., as compared to the original pathogenicity prediction neural network trained in step (170).

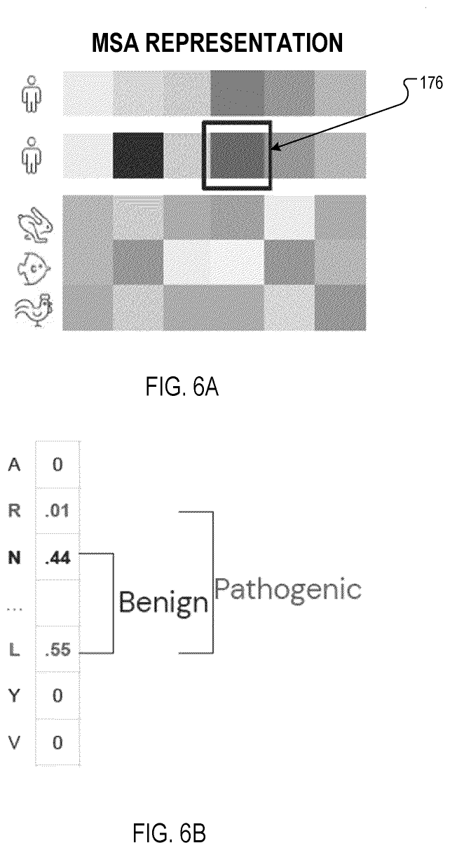

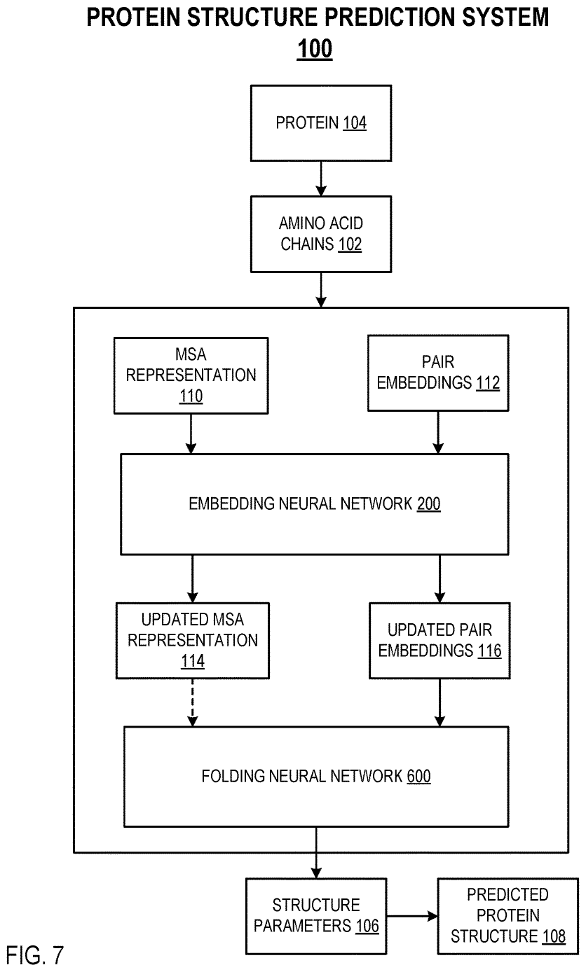

FIG. 6A illustrates an example of a MSA representation of an MSA for an protein. Each row of the MSA representation represents a respective MSA protein in the MSA. Each row includes a respective set of embeddings, including one embedding for each position in the amino acid sequence of the MSA protein represented by the row. The second row of the MSA representation represents the protein, and the embedding 176 represents a mutation position in the amino acid sequence of the protein and is masked in the example MSA representation. A pathogenicity prediction neural network can process the MSA representation to generate a pathogenicity score characterizing a likelihood of pathogenicity of a mutation to the mutation position in the amino acid sequence of the protein.

FIG. 6B illustrates an example of a score distribution over a set of amino acids generated by the pathogenicity prediction neural network. In FIG. 6B amino acids are designated using their 1-letter abbreviations. Amino acid L is the original amino acid at a mutation position in the amino acid sequence of a protein. A pathogenicity prediction system can evaluate the likelihood of a mutation from amino acid L to amino acid N at the mutation position being pathogenic based on a difference between the score for amino acid L (0.55) and the score for amino acid N (0.44) under the score distribution. The pathogenicity prediction system can evaluate the likelihood of a mutation from amino acid L to amino acid R at the mutation position based on a difference between the score for amino acid L (0.55) and the score for amino acid R (0.01) under the score distribution. The score distribution suggests that a mutation to amino acid R is more likely to be pathogenic than a mutation to amino acid N (the L-N difference is smaller than the L-R difference).

FIG. 7 shows an example protein structure prediction system 100. The protein structure prediction system 100 is an example of a system implemented as computer programs on one or more computers in one or more locations in which the systems, components, and techniques described below are implemented.

The system 100 is configured to process data defining one or more amino acid chains 102 of a protein 104 to generate a set of structure parameters 106 that define a predicted protein structure 108, i.e., a prediction of the structure of the protein 104. That is, the predicted structure 108 of the protein 104 can be defined by a set of structure parameters 106 that collectively define a predicted three-dimensional structure of the protein after the protein undergoes protein folding.

The structure parameters 106 defining the predicted protein structure 108 may be as previously described. For example they may include, e.g., location parameters and rotation parameters for each amino acid in the protein 104, a distance map that characterizes estimated distances between each pair of amino acids in the protein, a respective spatial location of each atom or backbone atom in each amino acid in the structure of the protein, or a combination thereof, as described above.

To generate the structure parameters 106 defining the predicted protein structure 108, the system 100 generates: (i) a multiple sequence alignment (MSA) representation 110 for the protein, and (ii) a set of “pair” embeddings 112 for the protein, as will be described in more detail next.

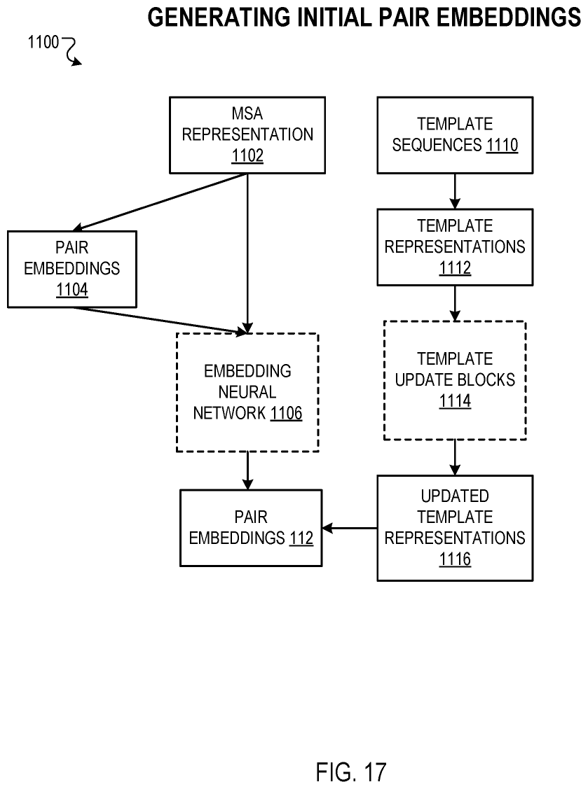

The MSA representation 110 for the protein includes a respective representation of a MSA for each amino acid chain in the protein. A MSA representation for an amino acid chain in the protein can be represented as a M×N array of embeddings (i.e., a 2-D array of embeddings having M rows and N columns), where N is the number of amino acids in the amino acid chain. Each row of the MSA representation can correspond to a respective MSA sequence for the amino acid chain in the protein. An example process for generating a MSA representation for an amino acid chain in the protein is described with reference to FIG. 16.

The system 100 generates the MSA representation 110 for the protein 104 from the MSA representations for the amino acid chains in the protein.

If the protein includes only a single amino acid chain, then the system 100 can identify the MSA representation 110 for the protein 104 as being the MSA representation for the single amino acid chain in the protein.

If the protein includes multiple amino acid chains, then the system 100 can generate the MSA representation 110 for the protein by assembling the MSA representations for the amino acid chains in the protein into a block diagonal 2-D array of embeddings, i.e., where the MSA representations for the amino acid chains in the protein form the blocks on the diagonal. The system 100 can initialize the embeddings at each position in the 2-D array outside the blocks on the diagonal to be a default embedding, e.g., a vector of zeros. The amino acid chains in the protein can be assigned an arbitrary ordering, and the MSA representations of the amino acid chains in the protein can be ordered accordingly in the block diagonal matrix. For example, the MSA representation for the first amino acid chain (i.e., according to the ordering) can be the first block on the diagonal, the MSA representation for the second amino acid chain can be the second block on the diagonal, and so on.

Generally, the MSA representation 110 for the protein can be represented as a 2-D array of embeddings. Throughout this specification, a “row” of the MSA representation for the protein refers to a row of a 2-D array of embeddings defining the MSA representation for the protein. Similarly, a “column” of the MSA representation for the protein refers to a column of a 2-D array of embeddings defining the MSA representation for the protein.

The set of pair embeddings 112 includes a respective pair embedding corresponding to each pair of amino acids in the protein 104. In general a pair embedding represents i.e. encodes information about the relationship between a pair of amino acids in the protein. A pair of amino acids refers to an ordered tuple that includes a first amino acid and a second amino acid in the protein, i.e., such that the set of possible pairs of amino acids in the protein is given by:

{(Aᵢ,Aⱼ):1≤i,j≤N} (1)

where N is the number of amino acids in the protein, i, j∈{1, . . . , N} index the amino acids in the protein, Aᵢ is the amino acid in the protein indexed by i, and Aⱼ is the amino acid in the protein indexed by j. If the protein includes multiple amino acid chains, then the amino acids in the protein can be sequentially indexed from {1, . . . , N} in accordance with the ordering of the amino acid chains in the protein. That is, the amino acids from the first amino acid chain are sequentially indexed first, followed by the amino acids from the second amino acid chain, followed by the amino acids from the third amino acid chain, and so on. The set of pair embeddings 112 can be represented as a 2-D, N×N array of pair embeddings, e.g., where the rows of the 2-D array are indexed by i∈{1, . . . , N}, the columns of the 2-D array are indexed by j∈{1, . . . , N}, and position (i, j) in the 2-D array is occupied by the pair embedding for the pair of amino acids (Aᵢ, Aⱼ).

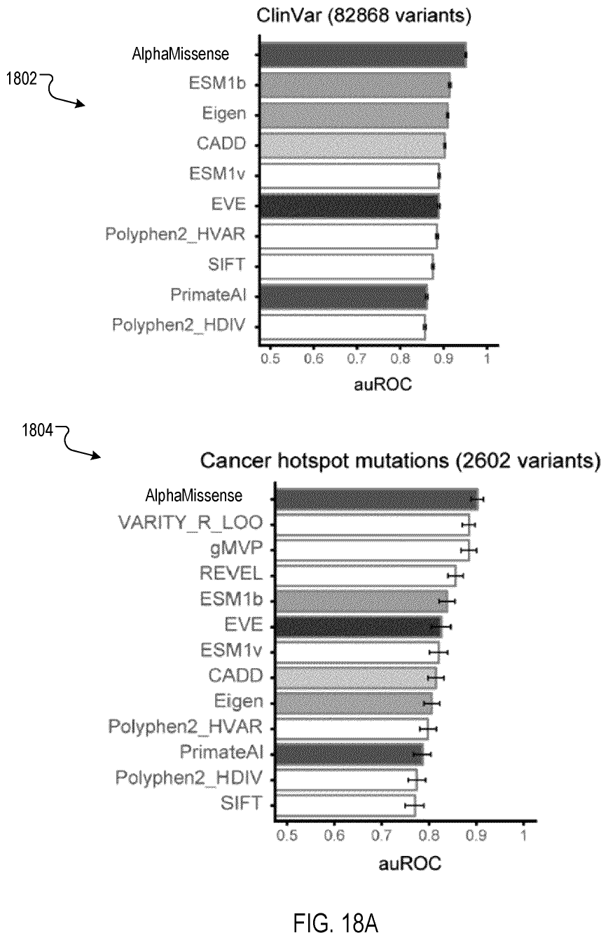

An example process for generating (initializing) a respective pair embedding corresponding to each pair of amino acids in the protein is described with reference to FIG. 17.

The system 100 generates the structure parameters 106 defining the predicted protein structure 108 using both the MSA representation 110 and the pair embeddings 112, because both have complementary properties. The structure of the MSA representation 110 can explicitly depend on the number of amino acid chains in the MSAs corresponding to each amino acid chain in the protein. Therefore, the MSA representation 110 may be inappropriate for use in directly predicting the protein structure, because the protein structure 108 has no explicit dependence on the number of amino acids chains in the MSAs. In contrast, the pair embeddings 112 characterize relationships between respective pairs of amino acids in the protein 104 and are expressed without explicit reference to the MSAs, and are therefore a convenient and effective data representation for use in predicting the protein structure 108.

The system 100 processes the MSA representation 110 and the pair embeddings 112 using an embedding neural network 200, in accordance with the values of a set of parameters of the embedding neural network 200, to update the MSA representation 110 and the pair embeddings 112. That is, the embedding neural network 200 processes the MSA representation 110 and the pair embeddings 112 to generate an updated MSA representation 114 and updated pair embeddings 116.

The embedding neural network 200 updates the MSA representation 110 and the pair embeddings 112 by sharing information between the MSA representation 110 and the pair embeddings 112. More specifically, the embedding neural network 200 alternates between updating the current MSA representation 110 based on the current pair embeddings 112, and updating the current pair embeddings 112 based on the current MSA representation 110.

An example architecture of the embedding neural network 200 is described in more detail with reference to FIG. 8.

The system 100 generates a network input for a folding neural network 600 from the updated pair embeddings 116, the updated MSA representation 114, or both, and processes the network input using the folding neural network 600 to generate the structure parameters 106 defining the predicted protein structure.

In some implementations, the folding neural network 600 processes the updated pair embeddings 116 to generate a distance map that includes, for each pair of amino acids in the protein, a probability distribution over a set of possible distances between the pair of amino acids in the protein structure. For example, to generate the probability distribution over the set of possible distances between a pair of amino acids in the protein structure, the folding neural network may apply one or more fully-connected neural network layers to an updated pair embedding 116 corresponding to the pair of amino acids.

In some implementations, the folding neural network 600 generates the structure parameters 106 by processing a network input derived from both the updated MSA representation 114 and the updated pair embeddings 116 using a geometric attention operation that explicitly reasons about the 3-D geometry of the amino acids in the protein structure. An example architecture of the folding neural network 600 that implements a geometric attention mechanism is described with reference to FIG. 12.

A training engine may train the protein structure prediction system 100 from end-to-end to optimize an objective function referred to herein as a structure loss. The training engine may train the system 100 on a set of training data including multiple training examples. Each training example may specify: (i) a training input that includes an initial MSA representation and initial pair embeddings for a protein, and (ii) a target protein structure that should be generated by the system 100 by processing the training input. Target protein structures used for training the system 100 may be determined using experimental techniques, e.g., x-ray crystallography.

The structure loss may characterize a similarity between: (i) a predicted protein structure generated by the system 100, and (ii) the target protein structure that should have been generated by the system.

For example, if the predicted structure parameters define predicted location parameters and predicted rotation parameters for each amino acid in the protein, then the structure loss [symbol] may be given by:

$${ℒ}_{structuτe} = \frac{1}{{N}^{2}} {\sum }_{i , j = 1}^{N} {( 1 - \frac{\vert {t}_{i j} - \text{?} \vert}{A} )}_{+} \qquad ( 2 )$$

$${t}_{i j} = {R}_{i}^{- 1} ( {t}_{j} - {t}_{i} ) \qquad ( 3 )$$

$$\text{?} = {\tilde{R}}_{ι}^{- 1} ( \text{?} - \text{?} ) \qquad ( 4 )$$

where N is the number of amino acids in the protein, tᵢ denote the predicted location parameters for amino acid i, Rᵢ denotes a 3×3 rotation matrix specified by the predicted rotation parameters for amino acid i, [symbol] , are the target location parameters for amino acid i, \tilde{R}ₗ denotes a 3×3 rotation matrix specified by the target rotation parameters for amino acid i, A is a constant,

$${R}_{i}^{- 1}$$

refers to the inverse of the 3×3 rotation matrix specified by predicted rotation parameters Rᵢ,

$${\tilde{R}}_{ι}^{- 1}$$

refers to the inverse of the 3×3 rotation matrix specified by the target rotation parameters \tilde{R}ₗ, and (·)₊ denotes a rectified linear unit (ReLU) operation.

The structure loss defined with reference to equations (2)-(4) may be understood as averaging the loss |tᵢⱼ− [symbol] | over each pair of amino acids in the protein. The term ty defines the predicted spatial location of amino acid j in the predicted frame of reference of amino acid i, and [symbol] defines the actual spatial location of amino acid j in the actual frame of reference of amino acid i. These terms are sensitive to the predicted and actual rotations of amino acid i and j, and therefore carry richer information than loss terms that are only sensitive to the predicted and actual distances between amino acids.

As another example, if the predicted structure parameters define predicted spatial locations of each atom in each amino acid of the protein, then the structure loss may be an average error (e.g., squared-error) between: (i) the predicted spatial locations of the atoms, and (ii) the target (e.g., ground truth) spatial locations of the atoms.

Optimizing the structure loss encourages the system 100 to generate predicted protein structures that accurately approximate true protein structures.

In addition to optimizing the structure loss, the training engine may train the system 100 to optimize one or more auxiliary losses. The auxiliary losses may penalize predicted structures having characteristics that are unlikely to occur in the natural world, e.g., based on the bond angles and/or bond lengths of the bonds between the atoms in the amino acids in the predicted structures, or based on the proximity of the atoms in different amino acids in the predicted structures.

The training engine may train the structure prediction system 100 on the training data over multiple training iterations, e.g., using stochastic gradient descent training techniques.

FIG. 8 shows an example architecture of an embedding neural network 200 that is configured to process the MSA representation 110 and the pair embeddings 112 to generate the updated MSA representation 114 and the updated pair embeddings 116.

The embedding neural network 200 includes a sequence of update blocks 202-A-N. Throughout this specification, a “block” refers to a portion of a neural network, e.g., a subnetwork of the neural network that includes one or more neural network layers.

Each update block in the embedding neural network is configured to receive a block input that includes a MSA representation and a pair embedding, and to process the block input to generate a block output that includes an updated MSA representation and an updated pair embedding.

The embedding neural network 200 provides the MSA representation 110 and the pair embeddings 112 included in the network input of the embedding neural network 200 to the first update block (i.e., in the sequence of update blocks). The first update block processes the MSA representation 110 and the pair embeddings 112 to generate an updated MSA representation and updated pair embeddings.

For each update block after the first update block, the embedding neural network 200 provides the update block with the MSA representation and the pair embeddings generated by the preceding update block, and provides the updated MSA representation and the updated pair embeddings generated by the update block to the next update block.

The embedding neural network 200 gradually enriches the information content of the MSA representation 110 and the pair embeddings 112 by repeatedly updating them using the sequence of update blocks 202-A-N.

The embedding neural network 200 may provide the updated MSA representation 114 and the updated pair embeddings 116 generated by the final update block (i.e., in the sequence of update blocks) as the network output.

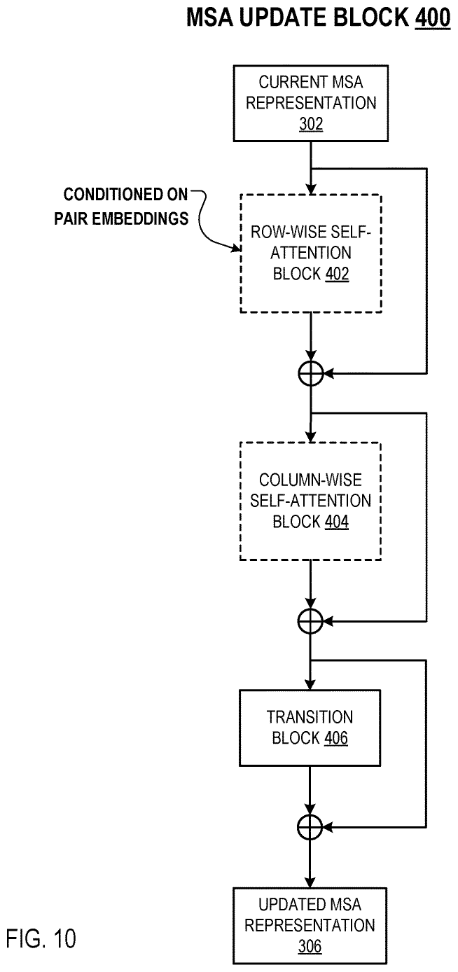

FIG. 9 shows an example architecture of an update block 300 of the embedding neural network 200, i.e., as described with reference to FIG. 8.

The update block 300 receives a block input that includes the current MSA representation 302 and the current pair embeddings 304, and processes the block input to generate the updated MSA representation 306 and the updated pair embeddings 308.

The update block 300 includes an MSA update block 400 and a pair update block 500.

The MSA update block 400 updates the current MSA representation 302 using the current pair embeddings 304, and the pair update block 500 updates the current pair embeddings 304 using the updated MSA representation 306 (i.e., that is generated by the MSA update block 400).

Generally, the MSA representation and the pair embeddings can encode complementary information. For example, the MSA representation can encode information about the correlations between the identities of the amino acids in different positions among a set of evolutionarily-related amino acid chains, and the pair embeddings can encode information about the inter-relationships between the amino acids in the protein. The MSA update block 400 enriches the information content of the MSA representation using complementary information encoded in the pair embeddings, and the pair update block 500 enriches the information content of the pair embeddings using complementary information encoded in the MSA representation. As a result of this enrichment, the updated MSA representation and the updated pair embedding encode information that is more relevant to predicting the protein structure.

The update block 300 is described herein as first updating the current MSA representation 302 using the current pair embeddings 304, and then updating the current pair embeddings 304 using the updated MSA representation 306. The description should not be understood as limiting the update block to performing operations in this sequence, e.g., the update block could first update the current pair embeddings using the current MSA representation, and then update the current MSA representation using the updated pair embeddings.

The update block 300 is described herein as including an MSA update block 400 (i.e., that updates the current MSA representation) and a pair update block 500 (i.e., that updates the current pair embeddings). The description should not be understood to limiting the update block 300 to include only one MSA update block or only one pair update block. For example, the update block 300 can include multiple MSA update blocks that update the MSA representation multiple times before the MSA representation is provided to a pair update block for use in updating the current pair embeddings. As another example, the update block 300 can include multiple pair update blocks that update the pair embeddings multiple times using the MSA representation.

The MSA update block 400 and the pair update block 500 can have any appropriate architectures that enable them to perform their described functions.

In some implementations, the MSA update block 400, the pair update block 500, or both, include one or more “self-attention” blocks. As used throughout this document, a self-attention block generally refers to a neural network block that updates a collection of embeddings, i.e., that receives a collection of embeddings and outputs updated embeddings. To update a given embedding, the self-attention block can determine a respective “attention weight” between the given embedding and each of one or more selected embeddings, and then update the given embedding using: (i) the attention weights, and (ii) the selected embeddings. For convenience, the self-attention block may be said to update the given embedding using attention “over” the selected embeddings.

For example, a self-attention block may receive a collection of input embeddings

$${\{ {x}_{i} \}}_{i = 1}^{N} ,$$

where N is the number of amino acids in the protein, and to update embedding xᵢ, the

$${[ {a}_{i , j} ]}_{j = 1}^{N}$$

where a_{i,j} denotes the attention self-attention block may determine attention weights weight between xᵢ and xⱼ, as:

$${[ {a}_{i , j} ]}_{j = 1}^{N} = softmax ( \frac{( {W}_{q} {x}_{i} ) {K}^{T}}{c} ) \qquad ( 5 )$$

$${K}^{T} = {[ {W}_{k} {x}_{j} ]}_{j = 1}^{N} \qquad ( 6 )$$

where W_{q} and Wₖ are learned parameter matrices, softmax(·) denotes a soft-max normalization operation, and c is a constant. Using the attention weights, the self-attention layer may update embedding xᵢ as:

$${x}_{i} ← {\sum }_{j = 1 … N} {a}_{i , j} \cdot ( {W}_{v} {x}_{j} ) \qquad ( 7 )$$

where Wᵥ is a learned parameter matrix. (W_{q}xᵢ can be referred to as the “query embedding” for input embedding xᵢ, Wₖxⱼ can be referred to as the “key embedding” for input embedding xᵢ, and Wᵥxⱼ can be referred to as the “value embedding” for input embedding xᵢ).

The parameter matrices W_{q} (the “query embedding matrix”), Wₖ (the “key embedding matrix”), and Wᵥ (the “value embedding matrix”) are trainable parameters of the self-attention block. The parameters of any self-attention blocks included in the MSA update block 400 and the pair update block 500 can be understood as being parameters of the update block 300 that can be trained as part of the end-to-end training of the protein structure prediction system 100 described with reference to FIG. 1. Generally, the (trained) parameters of the query, key, and value embedding matrices are different for different self-attention blocks, e.g., such that a self-attention block included in the MSA update block 400 can have different query, key, and value embedding matrices with different parameters than a self-attention block included in the pair update block 500.

In some implementations, the MSA update block 400, the pair update block 500, or both, include one or more self-attention blocks that are conditioned on the pair embeddings, i.e., that implement self-attention operations that are conditioned on the pair embeddings. To condition a self-attention operation on the pair embeddings, the self-attention block can process the pair embeddings to generate a respective “attention bias” corresponding to each attention weight. For example, in addition to determining the attention weights

$${[ {a}_{i , j} ]}_{j = 1}^{N}$$

in accordance with equations (5)-(6), the self-attention block can generate a corresponding set of attention biases

$${[ {b}_{i , j} ]}_{j = 1}^{N} ,$$

where b_{i,j} denotes the attention bias between xᵢ and xⱼ. The self-attention block biases can generate the attention bias b_{i,j} by applying a learned parameter matrix to the pair embedding h_{i,j}, i.e., for the pair of amino acids in the protein indexed by (i, j).

The self-attention block can determine a set of “biased attention weights”

$${[ {c}_{i , j} ]}_{j = 1}^{N} ,$$

where c_{i,j} denotes the biased attention weight between xᵢ and xⱼ, e.g., by summing (or otherwise combining) the attention weights and the attention biases. For example, the self-attention block can determine the biased attention weight c_{i,j} between embeddings xᵢ and xⱼ as:

c_{i,j}=a_{i,j}+b_{i,j}

where a_{i,j} is the attention weight between xᵢ and xⱼ and b_{i,j} is the attention bias between xᵢ and xⱼ. The self-attention block can update each input embedding xᵢ using the biased attention weights, e.g.:

$${x}_{i} ← {\sum }_{j = 1 .. N} {c}_{i , j} \cdot ( {W}_{v} {x}_{j} ) \qquad ( 8 )$$

where Wᵥ is a learned parameter matrix.

Generally, the pair embeddings encode information characterizing the structure of the protein and the relationships between the pairs of amino acids in the structure of the protein. Applying a self-attention operation that is conditioned on the pair embeddings to a set of input embeddings allows the input embeddings to be updated in a manner that is informed by the protein structural information encoded in the pair embeddings. The update blocks of the embedding neural network can use the self-attention blocks that are conditioned on the pair embeddings to update and enrich the MSA representation and the pair embeddings themselves.

Optionally, a self-attention block can have multiple “heads” that each generate a respective updated embedding corresponding to each input embedding, i.e., such that each input embedding is associated with multiple updated embeddings. For example, each head may generate updated embeddings in accordance with different values of the parameter matrices W_{q}, Wₖ, and Wᵥ that are described with reference to equations (5)-(7). A self-attention block with multiple heads can implement a “gating” operation to combine the updated embeddings generated by the heads for an input embedding, i.e., to generate a single updated embedding corresponding to each input embedding. For example, the self-attention block can process the input embeddings using one or more neural network layers (e.g., fully connected neural network layers) to generate a respective gating value for each head. The self-attention block can then combine the updated embeddings corresponding to an input embedding in accordance with the gating values. For example, the self-attention block can generate the updated embedding for an input embedding xᵢ as:

$${\sum }_{k = 1}^{K} {α}_{k} \cdot {x}_{i}^{next} \qquad ( 9 )$$

where k indexes the heads, αₖ is the gating value for head k, and

$${x}_{i}^{next}$$

is the updated embedding generated by head k for input embedding xᵢ.

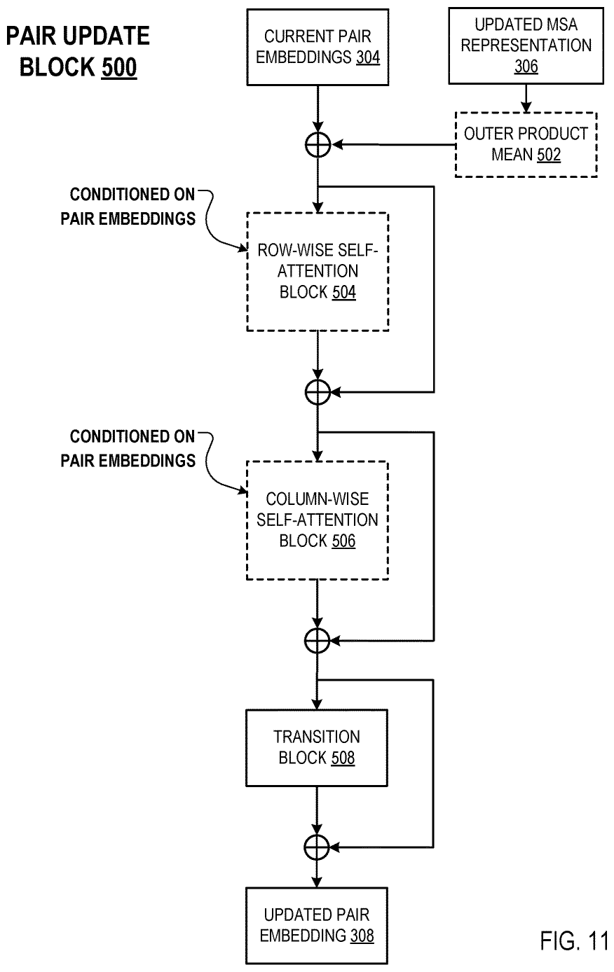

An example architecture of a MSA update block 400 that uses self-attention blocks conditioned on the pair embeddings is described with reference to FIG. 10. The example MSA update block described with reference to FIG. 10 updates the current MSA representation based on the current pair embeddings by processing the rows of the current MSA representation using a self-attention block that is conditioned on the current pair embeddings.

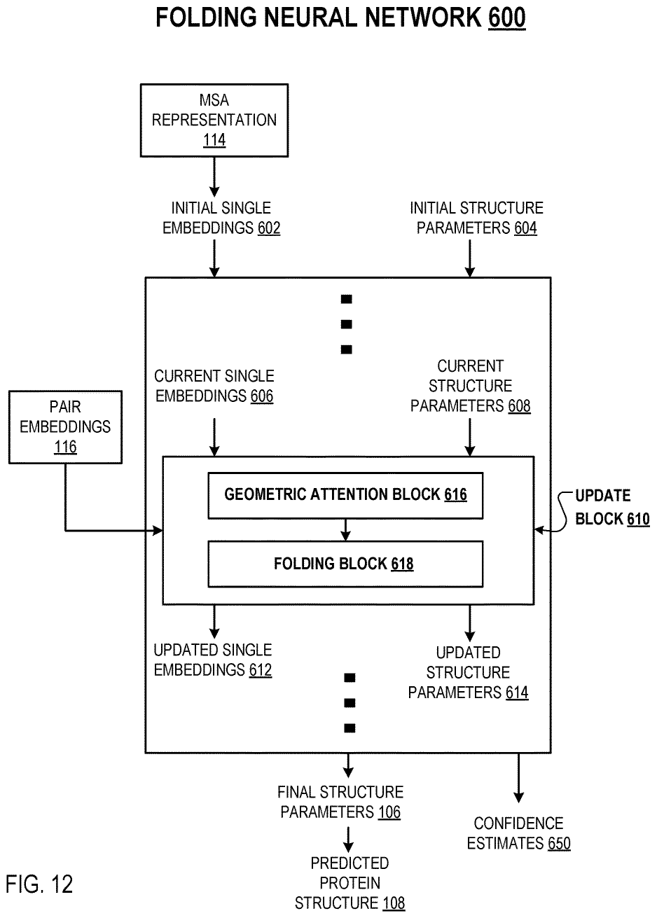

An example architecture of a pair update block 500 that uses self-attention blocks conditioned on the pair embeddings is described with reference to FIG. 11. The example pair update block described with reference to FIG. 11 updates the current pair embeddings based on the updated MSA representation by computing an outer product mean of the updated MSA representation, adding the result of the outer product mean to the current pair embeddings, and processing the current pair embeddings using self-attention blocks that are conditioned on the current pair embeddings.

FIG. 10 shows an example architecture of a MSA update block 400. The MSA update block 400 is configured to receive the current MSA representation 302, to update the current MSA representation 306 based (at least in part) on the current pair embedding.

To update the current MSA representation 302, the MSA update block 400 updates the embeddings in each row of the current MSA representation using a self-attention operation (i.e., a “row-wise” self-attention operation that operates only over embeddings in a particular row) that is conditioned on the current pair embeddings. More specifically, the MSA update block 400 provides the embeddings in each row of the current MSA representation 302 to a “row-wise” self-attention block 402 that is conditioned on the current pair embeddings, e.g., as described with reference to FIG. 9, to generate updated embeddings for each row of the current MSA representation 302. Optionally, the MSA update block can add the input to the row-wise self-attention block 402 to the output of the row-wise self-attention block 402. Conditioning the row-wise self-attention block 402 on the current pair embeddings enables the MSA update block 400 to enrich the current MSA representation 302 using information from the current pair embeddings.

The MSA update block then updates the embeddings in each column of the current MSA representation using a self-attention operation (i.e., a “column-wise” self-attention operation that operates only over embeddings in a particular column) that is not conditioned on the current pair embeddings. More specifically, the MSA update block 400 provides the embeddings in each column of the current MSA representation 302 to a “column-wise” self-attention block 404 that is not conditioned on the current pair embeddings to generate updated embeddings for each column of the current MSA representation 302. As a result of not being conditioned on the current pair embeddings, the column-wise self-attention block 404 generates updated embeddings for each column of the current MSA representation using attention weights (e.g., as described with reference to equations (5)-(6)) rather than biased attention weights (e.g., as described with reference to equation (8)). Optionally, the MSA update block can add the input to the column-wise self-attention block 404 to the output of the column-wise self-attention block 404.

The MSA update block then processes the current MSA representation 302 using a transition block, e.g., that applies one or more fully-connected neural network layers to the current MSA representation 302. Optionally, the MSA update block 400 can add the input to the transition block 406 to the output of the transition block 406.

The MSA update block can output the updated MSA representation 306 resulting from the operations performed by the row-wise self-attention block 402, the column-wise self-attention block 404, and the transition block 406.

FIG. 11 shows an example architecture of a pair update block 500. The pair update block 500 is configured to receive the current pair embeddings 304, and to update the current pair embeddings 304 based (at least in part) on the updated MSA representation 306.

To update the current pair embeddings 304, the pair update block 500 applies an outer product mean operation 502 to the updated MSA representation 306 and adds the result of the outer-product mean operation 502 to the current pair embeddings 304.

The outer product mean operation defines a sequence of operations that, when applied to an MSA representation represented as an M×N array of embeddings, generates an N×N array of embeddings, i.e, where N is the number of amino acids in the protein. The current pair embeddings 304 can also be represented as an N×N array of embeddings, and adding the result of the outer product mean 502 to the current pair embeddings 304 refers to summing the two N×N arrays of embeddings.

To compute the outer product mean, the pair update block generates a tensor A(·), e.g., given by:

$$A ( res 1 , res 2 , ch 1 , ch 2 ) = \frac{1}{\vert rows \vert} {\sum }_{rows} LeftAct ( row , res 1 , ch 1 ) \cdot RightAct ( row , res 2 , ch 2 )$$

where res1, res2∈{1, . . . , N}, ch1, ch2∈{1, . . . , C}, where C is the number of channels in each embedding of the MSA representation, |rows| is the number rows in the MSA representation, LeftAct(row,res1,ch1) is a linear operation (e.g., defined by a matrix multiplication) applied to the channel ch1 of the embedding of the MSA representation located at the row indexed by “row” and the column indexed by “res1”, and RightAct(row,res2,ch2) is a linear operation (e.g., defined by a matrix multiplication) applied to the channel ch2 of the embedding of the MSA representation located at the row indexed by “row” and the column indexed by “res2”. The result of the outer product mean is generated by flattening and linearly projecting the (ch1, ch2) dimensions of the tensor A. Optionally, the pair update block can perform one or more Layer Normalization operations (e.g., as described with reference to Jimmy Lei Ba et al., “Layer Normalization,” arXiv: 1607.06450) as part of computing the outer product mean.

Generally, the updated MSA representation 306 encodes information about the correlations between the identities of the amino acids in different positions among a set of evolutionarily-related amino acid chains. The information encoded in the updated MSA representation 306 is relevant to predicting the structure of the protein, and by incorporating the information encoded in the updated MSA representation into the current pair embeddings (i.e., by way of the outer product mean 502), the pair update block 500 can enhance the information content of the current pair embeddings.

After updating the current pair embeddings 304 using the updated MSA representation (i.e., by way of the outer product mean 502), the pair update block 500 updates the current pair embeddings in each row of an arrangement of the current pair embeddings into an N×N array using a self-attention operation (i.e., a “row-wise” self-attention operation) that is conditioned on the current pair embeddings. More specifically, the pair update block 500 provides each row of current pair embeddings to a “row-wise” self-attention block 504 that is also conditioned on the current pair embeddings, e.g., as described with reference to FIG. 9, to generate updated pair embeddings for each row. Optionally, the pair update block can add the input to the row-wise self-attention block 504 to the output of the row-wise self-attention block 504.

The pair update block 500 then updates the current pair embeddings in each column of the N×N array of current pair embeddings using a self-attention operation (i.e., a “column-wise” self-attention operation) that is also conditioned on the current pair embeddings. More specifically, the pair update block 500 provides each column of current pair embeddings to a “column-wise” self-attention block 506 that is also conditioned on the current pair embeddings to generate updated pair embeddings for each column. Optionally, the pair update block can add the input to the column-wise self-attention block 506 to the output of the column-wise self-attention block 506.

The pair update block 500 then processes the current pair embeddings using a transition block, e.g., that applies one or more fully-connected neural network layers to the current pair embeddings. Optionally, the pair update block 500 can add the input to the transition block 508 to the output of the transition block 508.

The pair update block can output the updated pair embeddings 308 resulting from the operations performed by the row-wise self-attention block 504, the column-wise self-attention block 506, and the transition block 508.

FIG. 12 shows an example architecture of a folding neural network 600 that generates a set of structure parameters 106 that define the predicted protein structure 108. The folding neural network 600 can be included in the protein structure prediction system 100 described with reference to FIG. 1.

The folding neural network 600 generates structure parameters that can include: (i) location parameters, and (ii) rotation parameters, for each amino acid in the protein. As described earlier, the location parameters for an amino acid may specify a predicted 3-D spatial location of a specified atom in the amino acid in the structure of the protein. The rotation parameters for an amino acid may specify the predicted “orientation” of the amino acid in the structure of the protein. More specifically, the rotation parameters may specify a 3-D spatial rotation operation that, if applied to the coordinate system of the location parameters, causes the three “main chain” atoms in the amino acid to assume fixed positions relative to the rotated coordinate system.

In implementations the folding neural network 600 receives an input derived from the final MSA representation, the final pair embeddings, or both and generates final values of the structure parameters 106 that define a predicted structure of the protein. For example the folding neural network 600 may receive an input that includes: (i) a respective pair embedding 116 for each pair of amino acids in the protein, (ii) initial values of a “single” embedding 602 for each amino acid in the protein, and (iii) initial values of structure parameters 604 for each amino acid in the protein. The folding neural network 600 processes the input to generate final values of the structure parameters 106 that collectively characterize the predicted structure 108 of the protein.

The protein structure prediction system 100 can provide the folding neural network 600 with the pair embeddings generated as an output of an embedding neural network, as described with reference to FIG. 1.

The protein structure prediction system 100 can generate the initial single embeddings 602 for the amino acids from the MSA representation 114, i.e., that is generated as an output of an embedding neural network, as described with reference to FIG. 1. For example, as described above, the MSA representation 114 can be represented as a 2-D array of embeddings having a number of columns equal to the number of amino acids in the protein, where each column is associated with a respective amino acid in the protein. The protein structure prediction system 100 can generate the initial single embedding for each amino acid in the protein by summing (or otherwise combining) the embeddings from the column of the MSA representation 114 that is associated with the amino acid. As another example, the protein structure prediction system 100 can generate the initial single embeddings for the amino acids in the protein by extracting the embeddings from a row of the MSA representation 114 that corresponds to the amino acid sequence of the protein whose structure is being estimated.

The protein structure prediction system 100 may generate the initial structure parameters 604 with default values, e.g., where the location parameters for each amino acid are initialized to the origin (e.g., [0,0,0] in a Cartesian coordinate system), and the rotation parameters for each amino acid are initialized to a 3×3 identity matrix.

The folding neural network 600 can generate the final structure parameters 106 by repeatedly updating the current values of the single embeddings 606 and the structure parameters 608, i.e., starting from their initial values. More specifically, the folding neural network 600 includes a sequence of update neural network blocks 610, where each update block 610 is configured to update the current single embeddings 606 (i.e., to generate updated single embeddings 612) and to update the current structure parameters 608 (i.e., to generate updated structure parameters 614). The folding neural network 600 may include other neural network layers or blocks in addition to the update blocks, e.g., that may be interleaved with the update blocks.

Each update block 610 can include: (i) a geometric attention block 616, and (ii) a folding block 618, each of which will be described in more detail next.

The geometric attention block 616 updates the current single embeddings using a “geometric” self-attention operation that explicitly reasons about the 3-D geometry of the amino acids in the structure of the protein, i.e., as defined by the structure parameters. More specifically, to update a given single embedding, the geometric attention block 616 determines a respective attention weight between the given single embedding and each of one or more selected single embeddings, where the attention weights depend on both the current single embeddings, the current structure parameters, and the pair embeddings. The geometric attention block 616 then updates the given single embedding using: (i) the attention weights, (ii) the selected single embeddings, and (iii) the current structure parameters.

To determine the attention weights, the geometric attention block 616 processes each current single embedding to generate a corresponding “symbolic query” embedding, “symbolic key” embedding, and “symbolic value” embedding. For example, the geometric attention block 616 may generate the symbolic query embedding qᵢ, symbolic key embedding kᵢ, and symbolic value embedding vᵢ for the single embedding hᵢ corresponding to the i-th amino acid as:

qᵢ=Linear(hᵢ) (10)

kᵢ=Linear(hᵢ) (11)

vᵢ=Linear(hᵢ) (12)

where Linear (.) refers to linear layers having independent learned parameter values.

The geometric attention block 616 additionally processes each current single embedding to generate a corresponding “geometric query” embedding, “geometric key” embedding, and “geometric value” embedding. The geometric query, geometric key, and geometric value embeddings for each single embedding are each 3-D points that are initially generated in the local reference frame of the corresponding amino acid, and then rotated and translated to a global reference frame using the structure parameters for the amino acid. For example, the geometric attention block 616 may generate the geometry query embedding

$${q}_{i}^{p} ,$$

geometric key embedding

$${k}_{i}^{p} ,$$

and geometric value embedding

$${v}_{i}^{p}$$

for the single embedding hᵢ corresponding to the i-th amino acid as:

$${q}_{i}^{p} = {R}_{i} \cdot {Linear}_{p} ( {h}_{i} ) + {t}_{i} \qquad ( 13 )$$

$${k}_{i}^{p} = {R}_{i} \cdot {Linear}_{p} ( {h}_{i} ) + {t}_{i} \qquad ( 14 )$$

$${v}_{i}^{p} = {R}_{i} \cdot {Linear}_{p} ( {h}_{i} ) + {t}_{i} \qquad ( 15 )$$

where Linearₚ(·) refers to linear layers having independent learned parameter values that project hᵢ to a 3-D point (the superscript p indicates that the quantity is a 3-D point), Rᵢ denotes the rotation matrix specified by the rotation parameters for the i-th amino acid, and tᵢ denotes the location parameters for the i-th amino acid.

To update the single embedding hᵢ corresponding to amino acid i, the geometric attention block 616 may generate attention weights

$${[ {a}_{j} ]}_{j = 1}^{N} ,$$

where N is the total number of amino acids in the protein and aⱼ is the attention weight between amino acid i and amino acid j, as:

$${[ {a}_{j} ]}_{j = 1}^{N} = softmax ( {[ \frac{{q}_{i} \cdot {k}_{j}}{\sqrt{m}} + α {\vert {q}_{i}^{p} - {k}_{j}^{p} \vert}_{2}^{2} + ( {b}_{i , j} \cdot w ) ]}_{j = 1}^{N} ) \qquad ( 16 )$$

where qᵢ denotes the symbolic query embedding for amino acid i, kⱼ denotes the symbolic key embedding for amino acid j, m denotes the dimensionality of qᵢ and kⱼ, α denotes a learned parameter,

$${q}_{i}^{p}$$

denotes the geometric query embedding for amino acid i,

$${k}_{j}^{p}$$

denotes the geometry key embedding for amino acid j, |·|₂ is an L₂ norm, and b_{i,j} is the pair embedding 116 corresponding to the pair of amino acids that includes amino acid i and amino acid j, and w is a learned weight vector (or some other learned projection operation).

Generally, the pair embedding for a pair of amino acids implicitly encodes information relating the relationship between the amino acids in the pair, e.g., the distance between the amino acids in the pair. By determining the attention weight between amino acid i and amino acid j based in part on the pair embedding for amino acids i and j, the folding neural network 600 enriches the attention weights with the information from the pair embedding and thereby improves the accuracy of the predicted folding structure.

In some implementations, the geometric attention block 616 generate multiple sets of geometric query embeddings, geometric key embeddings, and geometric value embeddings, and uses each generated set of geometric embeddings in determining the attention weights.

After generating the attention weights for the single embedding hᵢ corresponding to amino acid i, the geometric attention block 616 uses the attention weights to update the single embedding hᵢ. In particular, the geometric attention block 616 uses the attention weights to generate a “symbolic return” embedding and a “geometric return” embedding, and then updates the single embedding using the symbolic return embedding and the geometric return embedding. The geometric attention block 124 may generate the symbolic return embedding oᵢ for amino acid i, e.g., as:

$${o}_{i} = {\sum }_{j} {a}_{j} {v}_{j} \qquad ( 17 )$$

where

$${[ {a}_{j} ]}_{j = 1}^{N}$$

denote the attention weights (e.g., defined with reference to equation (16)) and each vⱼ denotes the symbolic value embedding for amino acid j. The geometric attention block 616 may generate the geometric return embedding

$${o}_{i}^{p}$$

for amnio acid i, e.g., as:

$${o}_{i}^{p} = {R}_{i}^{- 1} \cdot ( {\sum }_{j} {a}_{j} {v}_{j}^{p} - {t}_{i} ) \qquad ( 18 )$$

where the geometric return embedding

$${o}_{i}^{p}$$

is a 3-D point,

$${[ {a}_{j} ]}_{j = 1}^{N}$$

denote the attention weights (e.g., defined with reference to equation (16)),

$${R}_{i}^{- 1}$$

is inverse of the future matrix specified by the rotation parameters for amino acid i, and tᵢ are the location parameters for amino acid i. It can be appreciated that the geometric return embedding is initially generated in the global reference frame, and then rotated and translated to the local reference frame of the corresponding amino acid.

The geometric attention block 616 may update the single embedding hᵢ for amino acid i using the corresponding symbolic return embedding oᵢ (e.g., generated in accordance with equation (17)) and geometric return embedding

$${o}_{i}^{p}$$

(e.g., generated in accordance with equation (18)), e.g., as:

$${h}_{i}^{n e x t} = ( {h}_{i} + Linear ( {o}_{i} , {o}_{i}^{p} , \vert {o}_{i}^{p} \vert ) ) \qquad ( 19 )$$

where

$${h}_{i}^{n e x t}$$

is the updated single embedding for amino acid i, |·| is a norm, e.g., an L₂ norm, and LayerNorm(·) denotes a layer normalization operation, e.g., as described with reference to: J. L. Ba, J. R. Kiros, G. E. Hinton, “Layer Normalization,” arXiv:1607.06450 (2016).

Updating the single embeddings 606 of the amino acids using concrete 3-D geometric embeddings, e.g., as described with reference to equations (13)-(15), enables the geometric attention block 616 to reason about 3-D geometry in updating the single embeddings. Moreover, each update block updates the single embeddings and the structure parameters in a manner that is invariant to rotations and translations of the overall protein structure. For example, applying the same global rotation and translation operation to the initial structure parameters provided to the folding neural network 600 would cause the folding neural network 600 to generate a predicted structure that is globally rotated and translated in the same way, but otherwise the same. Therefore, global rotation and translation operations applied to the initial structure parameters would not affect the accuracy of the predicted protein structure generated by the folding neural network 600 starting from the initial structure parameters. The rotational and translational invariance of the representations generated by the folding neural network 600 facilitates training, e.g., because the folding neural network 600 automatically learns to generalize across all rotations and translations of protein structures.

The updated single embeddings for the amino acids may be further transformed by one or more additional neural network layers in the geometric attention block 616, e.g., linear neural network layers, before being provided to the folding block 618.

After the geometric attention block 616 updates the current single embeddings 606 for the amino acids, the folding block 618 updates the current structure parameters 608 using the updated single embeddings 612. For example, the folding block 618 may update the current location parameters tᵢ for amino acid i as:

$${t}_{i}^{n e x t} = {t}_{i} + Linear ( {h}_{i}^{n e x t} ) \qquad ( 20 )$$

where

$${t}_{i}^{n e x t}$$

are the updated location parameters, Linear(·) denotes a linear neural network layer, and

$${h}_{i}^{n e x t}$$

denotes the updated single embedding for amino acid i. In another example, the rotation parameters Rᵢ for amino acid i may specify a rotation matrix, and the folding block 618 may update the current rotation parameters Rᵢ as:

$${w}_{i} = Linear ( {h}_{i}^{n e x t} ) \qquad ( 21 )$$

$${R}_{i}^{n e x t} = {R}_{i} \cdot QuaternionToRotation ( 1 + {w}_{i} ) \qquad ( 22 )$$

where wᵢ is a three-dimensional vector, Linear(·) is a linear neural network layer,

$${h}_{i}^{n e x t}$$

is the updated single embedding for amino acid i, 1+wᵢ denotes a quaternion with real part 1 and imaginary part wᵢ, and QuaternionToRotation(·) denotes an operation that transforms a quaternion into an equivalent 3×3 rotation matrix. Updating the rotation parameters using equations (21)-(22) ensures that the updated rotation parameters define a valid rotation matrix, e.g., an orthonormal matrix with determinant one.

The folding neural network 600 may provide the updated structure parameters generated by the final update block 610 as the final structure parameters 106 that define the predicted protein structure 108. The folding neural network 600 may include any appropriate number of update blocks, e.g., 5 update blocks, 25 update blocks, or 125 update blocks. Optionally, each of the update blocks of the folding neural network may share a single set of parameter values that are jointly updated during training of the folding neural network. Sharing parameter values between the update blocks 610 reduces the number of trainable parameters of the folding neural network and may therefore facilitate effective training of the folding neural network, e.g., by stabilizing the training and reducing the likelihood of overfitting.

During training, a training engine can train the parameters of the structure prediction system, including the parameters of the folding neural network 600, based on a structure loss that evaluates the accuracy of the final structure parameters 106, as described above. In some implementations, the training engine can further evaluate an auxiliary structure loss for one or more of the update blocks 610 that precede the final update block (i.e., that produces the final structure parameters). The auxiliary structure loss for an update block evaluates the accuracy of the updated structure parameters generated by the update block.

Optionally, during training, the training engine can apply a “stop gradient” operation to prevent gradients from backpropagating through certain neural network parameters of each update block, e.g., the neural network parameters used to compute the updated rotation parameters (as described in equations (21)-(22)). Applying these stop gradient operations can improve the numerical stability of the gradients computed during training.

Generally, a similarity between the predicted protein structure 108 generated by the folding neural network 600 and the corresponding ground truth protein structure can be measured, e.g., by a similarity measure that assigns a respective accuracy score to each of multiple atoms in the predicted protein structure. For example, the similarity measure can assign a respective accuracy score to each carbon alpha atom in the predicted protein structure. The accuracy score for an atom in the predicted protein structure can characterize how closely the position of the atom in the predicted protein structure conforms with the actual position of the atom in the ground truth protein structure. An example of a similarity measure that can compare the predicted protein structure to the ground truth protein structure to generate accuracy scores for the atoms in predicted protein structure is the IDDT similarity measure described with reference to: V. Mariani et al., “IDDT: a local superposition-free score for comparing protein structures and models using distance difference tests,” Bioinformatics, 2013 Nov. 1; 29 (21) 2722-2728.

The folding neural network 600 can be configured to generate a respective confidence estimate 650 for each of one or more atoms in the predicted protein structure 108. The confidence estimate 650 for an atom in the predicted protein structure characterizes the predicted accuracy score (e.g., IDDT accuracy score) for the atom in the predicted protein structure, i.e., that would be generated by a similarity measure that compares the predicted protein structure to the (potentially unknown) ground truth protein structure. In one example, the confidence estimate 650 for an atom in the predicted protein structure can define a discrete probability distribution over a set of intervals that form a partition of a range of possible values for the accuracy score for the atom. The discrete probability distribution can associate a respective probability with each of the intervals that defines the likelihood that the actual accuracy score is included in the interval. For example, range of possible values of the accuracy score may be [0, 100], and the confidence estimate 650 may define a probability distribution over the set of intervals {[0, 2), [2,4), . . . , [98,100]}. In another example, the confidence estimate 650 for an atom in the predicted protein structure can be a numerical value, i.e., that directly predicts the accuracy score for the atom.

In some implementations, the folding neural network 600 generates a respective confidence estimate 650 for a specified atom (e.g., the alpha carbon atom) in each amino acid of the protein. The folding neural network 600 can generate the confidence estimate 650 for the specified atom in an amino acid in the protein, e.g., by processing the updated single embedding for the amino acid that is generated by the last update block in the folding neural network using one or more neural network layers, e.g., fully-connected layers.

The structure prediction system can generate a respective confidence score corresponding to each amino acid in the protein based on the confidence estimates 650 for the atoms in the predicted protein structure. For example, the structure prediction system can generate a confidence score for an amino acid as the expected value of a probability distribution over possible values of the accuracy score for the alpha carbon atom in the amino acid.

The structure prediction system can generate a confidence score for the entire predicted structure, e.g., as an average of the confidence scores for the amino acids in the protein.

During training of the structure prediction system, a training engine can adjust the parameter values of the structure prediction system by backpropagating gradients of an auxiliary loss that measures an error between: (i) confidence estimates generated by the folding neural network 600, and (ii) accuracy scores generated by comparing the predicted protein structure to the ground truth protein structure. The error may be, e.g., a cross-entropy error.

Confidence estimates generated by structure prediction systems can be used in a variety of ways. For example, confidence estimates for atoms in the predicted protein structure can indicate which parts of the structure have been reliably estimated are therefore suitable for further downstream processing or analysis. As another example, per-protein confidence scores can be used to rank a set of predictions for the structure of a protein, e.g., that have been generated by the same structure prediction system by processing different inputs characterizing the same protein, or that have been generated by different structure prediction systems.

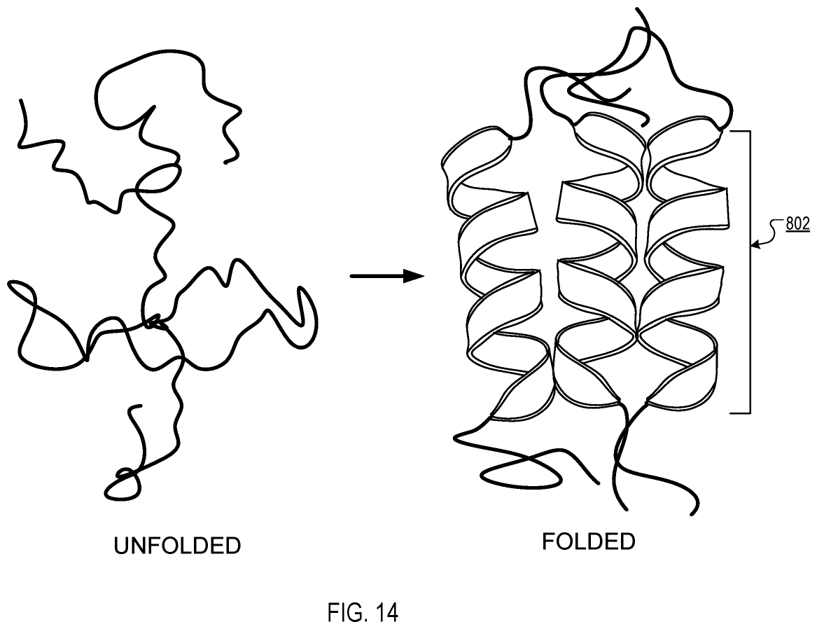

The location and rotation parameters specified by the structure parameters 106 can define the spatial locations (e.g., in [x, y, z] Cartesian coordinates) of the main chain atoms in the amino acids of the protein. However, the structure parameters 106 do not necessarily define the spatial locations of the remaining atoms in the amino acids of the protein, e.g., the atoms in the side chains of the amino acids. In particular, the spatial locations of the remaining atoms in an amino acid depend on the values of the torsion angles between the bonds in the amino acid, e.g., the omega-angle, the phi-angle, the psi-angle, the chi1-angle, the chi2-angle, the chi3-angle, and the chi4 angle, as illustrated with reference to FIG. 13.

Optionally, one or more of the update blocks 610 of the folding neural network 600 can generate an output that defines a respective predicted spatial location for each atom in each amino acid of the protein. To generate the predicted spatial locations for the atoms in an amino acid, the update block can process the updated single embedding for the amino acid using one or more neural network layers to generate predicted values of the torsion angles of the bonds between the atoms in the amino acid. The neural network layers may be, e.g., fully-connected neural network layers embedded with residual connections. Each torsion angle may be represented, e.g., as a 2-D vector.

The update block can determine the spatial locations of the atoms in an amino acid based on: (i) the values of the torsion angles for the amino acid, and (ii) the updated structure parameters (e.g., location and rotation parameters) for the amino acid. For example, the update block can process the torsion angles in accordance with a predefined function to generate the spatial locations of the atoms in the amino acid in a local reference frame of the amino acid. The update block can generate the spatial locations of the atoms in the amino acid in a global reference frame (i.e., that is common to all the amino acids in the protein) by rotating and translating the spatial locations of the atoms in accordance with the updated structure parameters for the amino acid. For example, the update block can determine the spatial location of an atom in the global reference frame by applying the rotation operation defined by the updated rotation parameters to the spatial location of the atom in the local reference frame to generate a rotated spatial location, and then apply the translation operation defined by the updated location parameters to the rotated spatial location.

In some implementations, alternatively to or in combination with outputting the final structure parameters, the folding neural network 600 outputs the predicted spatial locations of the atoms in the amino acids of the protein that are generated by the final update block.

The folding neural network 600 described with reference to FIG. 12 is characterized herein as receiving an input that is based on an MSA representation 114 and pair embeddings 116 that are generated by an embedding neural network, e.g., as described with reference to FIG. 8. In general, however, the inputs to the folding neural network (e.g., the single embeddings 602 and the pair embeddings 116) can be generated using any appropriate technique. Moreover, various aspects of the operations performed by the folding neural network (e.g., predicting spatial locations for the atoms in each amino acid of the protein) can be performed by other folding neural networks, e.g., with different architectures that receive different inputs.

FIG. 13 illustrates the torsion angles between the bonds in the amino acid, e.g., the omega-angle, the phi-angle, the psi-angle, the chi1-angle, the chi2-angle, the chi3-angle, the chi4 angle, and the chi5 angle.

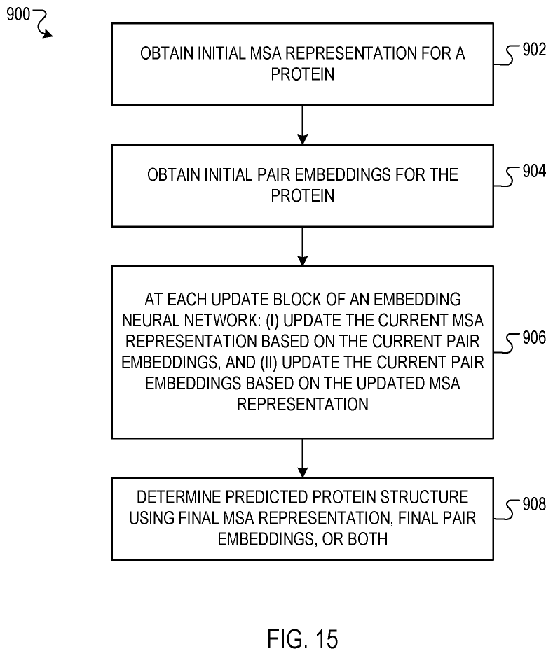

FIG. 14 is an illustration of an unfolded protein and a folded protein. The unfolded protein is a random coil of amino acids. The unfolded protein undergoes protein folding and folds into a 3D configuration. Protein structures often include stable local folding patterns such alpha helices (e.g., as depicted by 802) and beta sheets.

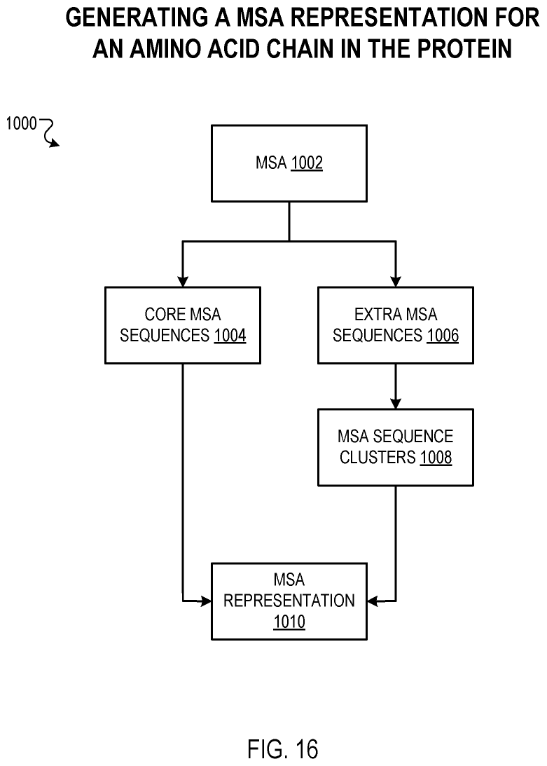

FIG. 15 is a flow diagram of an example process 900 for predicting the structure of a protein that includes one or more chains, where each chain specifies a sequence of amino acids. For convenience, the process 900 will be described as being performed by a system of one or more computers located in one or more locations. For example, a protein structure prediction system, e.g., the protein structure prediction system 100 of FIG. 1, appropriately programmed in accordance with this specification, can perform the process 900.

The system obtains an initial multiple sequence alignment (MSA) representation that represents a respective MSA corresponding to each chain in the protein (902).

The system obtains a respective initial pair embedding for each pair of amino acids in the protein (904).

The system processes an input including the initial MSA representation and the initial pair embeddings using an embedding neural network to generate an output that includes a final MSA representation and a respective final pair embedding for each pair of amino acids in the protein.

The embedding neural network includes a sequence of update blocks. Each update block has a respective set of update block parameters and is configured to receive a current MSA representation and a respective current pair embedding for each pair of amino acids in the protein. Each update block: (i) updates the current MSA representation based on the current pair embeddings, and (ii) updates the current pair embeddings based on the updated MSA representation (906).

The system determines a predicted structure of the protein based using the final MSA representation, the final pair embeddings, or both (908).

FIG. 16 shows an example process 1000 for generating a MSA representation 1010 for an amino acid chain in the protein. The protein structure prediction system 100, described with reference to FIG. 1, can implement the operations of the process 1000.

To generate the MSA representation 100 for the amino acid chain in the protein, the system 100 obtains a MSA 1002 for the protein that may include, e.g., thousands of MSA sequences.

The system 100 divides the set of MSA sequences into a set of “core” MSA sequences 1004 and a set of “extra” MSA sequences 1006. The set of core MSA sequences can be smaller (e.g., by an order of magnitude) than the set of extra MSA sequences 1006. The system 100 can divide the set of MSA sequences into core MSA sequences 1004 and extra MSA sequences 1006, e.g., by randomly selecting a predetermined number of the MSA sequences as core MSA sequences, and identifying the remaining MSA sequences as extra MSA sequences 1006.

For each extra MSA sequence 1006, the system 100 can determine a respective similarity measure (e.g., based on a Hamming distance) between the extra MSA sequence and each core MSA sequence 1004. The system 100 can then associate each extra MSA sequence 1006 with the corresponding core MSA sequence 1004 to which the extra MSA sequence 1006 is most similar (i.e., according to the similarity measure). The set of extra MSA sequences 1006 associated with a core MSA sequence 1004 can be referred to as a “MSA sequence cluster” 1008. That is, the system 100 determines a respective MSA sequence cluster 1008 corresponding to each core MSA sequence 1004, where the MSA sequence cluster 1008 corresponding to a core MSA sequence 1004 includes the set of extra MSA sequences 1006 that are most similar to the core MSA sequence 1004.

The system 100 can generate the MSA representation for the amino acid chain in the protein based on the core MSA sequences and the MSA sequence clusters 1008. The MSA representation 1010 can be represented by an M×N array of embeddings, where M is the number of core MSA sequences (i.e., such that each core MSA sequence is associated with a respective row of the MSA representation), and N is the number of amino acids in the amino acid chain. The embeddings in the MSA representation can be indexed by (i, j)∈{(i, j): i=1, . . . , M, j=1, . . . , N}.

To generate the embedding at position (i, j) in the MSA representation 1010, the system 100 can obtain an embedding (e.g., a one-hot embedding) defining the identity of the amino acid at position j in core MSA sequence i. The system 100 can also determine a probability distribution over the set of possible amino acids based on the relative frequency of occurrence of each possible amino acid at position j in the extra MSA sequences 1006 in the MSA sequence cluster 1008 corresponding to core MSA sequence i. The system 100 can then determine the embedding at position (i, j) in the MSA representation by combining (e.g., concatenating): (i) the embedding defining the identity of the amino acid at position j in core MSA sequence i, and (ii) the probability distribution over possible amino acids corresponding to position j in core MSA sequence i.

In some cases, the (ground truth) protein structure may be known for one or more of the core MSA sequences. In particular, for one or more of the core MSA sequences, the values of the torsion angles between the bonds in the amino acids in the core MSA sequence (e.g., the omega-angle, the phi-angle, the psi-angle, etc.) may be known. If the values of the torsion angles for the amino acids in core MSA sequence i are known, then the system 100 can generate the embedding at position (i, j) in the MSA representation based at least in part on the values of the torsion angles for amino acid j in core MSA sequence i. For example, the system can generate an embedding of the values of the torsion angles using one or more neural network layers, and the concatenate the embedding of the values of the torsion angles to the embedding at position (i, j) in the MSA representation.

FIG. 17 shows an example process 1100 for generating (initializing) a respective pair embedding 112 for each pair of amino acids in the protein. The protein structure prediction system 100, described with reference to FIG. 1, can implement the operations of the process 1100.

The system 100 can generate the pair embeddings 112 using an MSA representation 1102 of the protein. Generating a MSA representation for the protein is described in more detail with reference to FIG. 7 and FIG. 16. The system 100 can generate the MSA representation 1102 for the protein based on a respective MSA representation for each amino acid chain in the protein. To generate the MSA representation for an amino acid chain in the protein, the system 100 can use more MSA sequences (e.g., by an order of magnitude) than were used to generate the MSA representation 110 described with reference to FIG. 1. Therefore, the MSA representation 1102 used by the system 100 to generate the pair embeddings 112 may have more rows (e.g., by an order of magnitude) than the MSA representation 110 described with reference to FIG. 1. In some implementations, the system 100 can use the extra MSA sequences 1006 described with reference to FIG. 16 to generate the MSA representation 1102.

After generating the MSA representation 1102, the system 100 processes the MSA representation 1102 to generate pair embeddings 1104 from the MSA representation 1102, e.g., by applying an outer product mean operation to the MSA representation 1102, and identifying the pair embeddings 1104 as the result of the outer product mean operation.

The system 100 processes the MSA representation 1102 and the pair embeddings 1104 using an embedding neural network 1106. The embedding neural network 1106 can update the MSA representation 1102 and the pair embeddings 1104 by sharing information between the MSA representation 1102 and the pair embeddings 1104. More specifically, the embedding neural network 1106 can alternate between updating the MSA representation 1102 based on the pair embeddings 1104, and updating the pair embeddings 1104 based on the MSA representation 1102.

The embedding neural network 1106 can have an architecture based on the embedding neural network architecture described with reference to FIGS. 2-5, i.e., that updates the pair embeddings 1104 and the MSA representation 1102 using row-wise and column-wise self-attention blocks.

In some implementations, the embedding neural network 1106 can update the embeddings in each column of the MSA representation 1102 using a column-wise “global” self-attention operation. More specifically, the embedding neural network 1106 can provide the embeddings in each column of the MSA representation to a column-wise global self-attention block to generate updated embeddings for each column of the current MSA representation. To implement global column-wise self-attention, the self-attention block can generate a respective query embedding for each embedding in a column, and then average the query embeddings to generate a single “global” query embedding for the column. The column-wise self-attention block then uses the single global query embedding to perform the self-attention operation, which can reduce the complexity of the self-attention operation from quadratic (i.e., in the number of embeddings per column) to linear. Using a global self-attention operation can reduce the computational complexity of the column-wise self-attention operation to enable the column-wise self-attention operation to be performed on columns of the MSA representation 1102 that include large numbers (e.g., thousands) of embeddings.

After updating the pair embeddings 1104 and the MSA representation 1102 using the embedding neural network 1106, the system 100 can identify the pair embeddings 112 as the updated pair embeddings generated by the embedding neural network 1106. The system 100 can discard the updated MSA representation generated by the embedding neural network 1106, or use it any appropriate way.

As part of generating the pair embeddings 112, the system 100 can include relative position encoding information in the respective pair embedding corresponding to each pair of amino acids in the protein. The system can include the relative position encoding information for a pair of amino acids that are included in the same amino acid chain in the corresponding pair embedding by: computing the signed difference representing the number of amino acids separating the pair of amino acids in the amino acid chain, clipping the result to a predefined interval, representing the clipped value using a one-hot encoding vector, applying a linear transformation to the one-hot encoding vector, and adding the result of the linear transformation to the corresponding pair embedding. The system can include the relative position encoding information for a pair of amino acids that are not included in the same amino acid chain in the corresponding pair embedding by adding a default encoding vector to the corresponding pair embedding which indicates that the pair of amino acids are not included in the same amino acid chain.

The system 100 can also generate the pair embeddings 112 based at least in part on a set of one or more template sequences 1110. Each template sequence 1110 is an MSA sequence for an amino acid chain in the protein where the folded structure of the template sequence 1110 is known, e.g., from physical experiments.

The system 100 can generate a respective template representation 1112 of each template sequence 1110. A template representation 1112 of a template sequence 1110 includes a respective embedding corresponding to each pair of amino acids in the template sequence, e.g., such that a template representation 1112 of a template sequence of length n (i.e., with n amino acids) can be represented as an n×n array of embeddings. The system 100 generate the embedding at position (i, j) in the template representation 1112 of a template sequence 1110 based on, e.g.: (i) respective embeddings (e.g., one-hot embeddings) representing the identities of the amino acid at position i and position j in the template sequence, (ii) a unit vector defined by the difference in spatial positions of the respective carbon alpha atoms in the amino acids at position i and j in the template sequence, i.e., in the folded structure of the template sequence, where the unit vector is computed in the frame of reference of amino acid i or amino acid j, and (iii) a discretized/binned representation of the distance between the spatial positions of the respective carbon alpha atoms in the amino acids at position i and j in the template sequence.

The system 100 can process each template representation using a sequence of one or more template update blocks 1114 to generate a respective updated template representation 1116 corresponding to each template sequence 1110. The template update blocks can include, e.g., row-wise self-attention blocks (e.g., that update the embeddings in each row of the template representations), column-wise self-attention blocks (e.g., that update the embeddings in each column of the template representations), and transition blocks (e.g., that apply one or more neural network layers to each of the embeddings in the template representations).

After generating the updated template representations 1116, the system 100 uses the updated template representations 1116 to update the pair embeddings 112. For example, the system 100 can update the respective pair embedding 112 at each position (i, j) using “cross-attention” over the embeddings at the corresponding (i, j) positions of the updated template representations 1116. In a cross-attention operation to update the pair embedding 112 at position (i, j), the query embedding is generated from the pair embedding at position (i, j), and the key and value embeddings are generated from the embeddings at the corresponding (i, j) positions of the updated template representations 1116.

Updating the pair embeddings 112 using the template sequences 1110 enables the system 100 to enrich the pair embeddings with information characterizing the protein structures of the evolutionarily related template sequences 1110, thereby enhancing the information content of the pair embeddings 112, and improving the accuracy of protein structures predicted using the pair embeddings.

As previously mentioned, the pathogenicity prediction system 120 described in this specification has a variety of possible applications, including using the pathogenicity score to identify a cause of disease in an animal or plant subject.

In another example application the system is used for obtaining a mutated protein for pest control. This can comprise determining the pathogenicity score for a plurality of mutations of a protein, e.g. with respect to a reference amino acid sequence, each mutation defining a respective mutated protein. Then one of the mutations can be selected, based on the pathogenicity scores for the mutations, to define the mutated protein for use in pest control. For example the pathogenicity scores can be ranked and the most pathogenic mutation according to the scores can be selected e.g. one with a highest score. As another example a mutation with an intermediate pathogenicity score can be selected, e.g. so as to facilitate spread of the mutation through a population of the pests. To use the mutated protein for pest control the pest may be bred to produce the selected mutated protein.

In another example application the system is used for screening one or more living organisms, e.g. animals (including humans) or plants, for the presence of a protein that has a pathogenic mutation. This can comprise obtaining the amino acid sequence of the protein for each of the organisms, e.g. by gene sequencing as previously described. In general some of these sequences will be for mutated versions of the protein. For each of the obtained amino acid sequences that includes a mutation, e.g. in comparison with a reference amino acid sequences of a reference protein, the system determines the pathogenicity score for the mutation. Each determined pathogenicity score can then be processed to determine whether the mutation is pathogenic, e.g. by classifying the pathogenicity score as benign or pathogenic. The pathogenicity score, or the classification, can e.g. provide input to a clinical decision as to whether or how to provide treatment.

In another example application the system is used for determining a degree of pathogenicity of a bacterium or virus to a living organism. This can comprise obtaining the amino acid sequence of a manufactured protein, where the manufactured protein is made inside the living organism, e.g. by the living organism or by the bacterium, as a result of infection of the living organism by the bacterium or virus. The manufactured protein may be a toxin generated by the bacterium or a protein manufactured by the living organism, e.g. a viral protein and can be a mutation of a naturally occurring protein in the organism. The pathogenicity score of the mutation of the naturally occurring protein can be determined and used to determine a degree of pathogenicity of the bacterium or virus. For example the pathogenicity score may be classified as benign or pathogenic, or a relative pathogenicity of different strains of the bacterium or virus can be compared. This can be used, for example, to determine whether a proportion of pathogenic strains in a population is increasing or decreasing, e.g. to trigger an alert; or to select a bacterium or virus according to the determined degree of pathogenicity for manufacturing a vaccine, e.g. to identify a strain of a bacterium or virus with a relatively lower pathogenicity for manufacturing the vaccine.

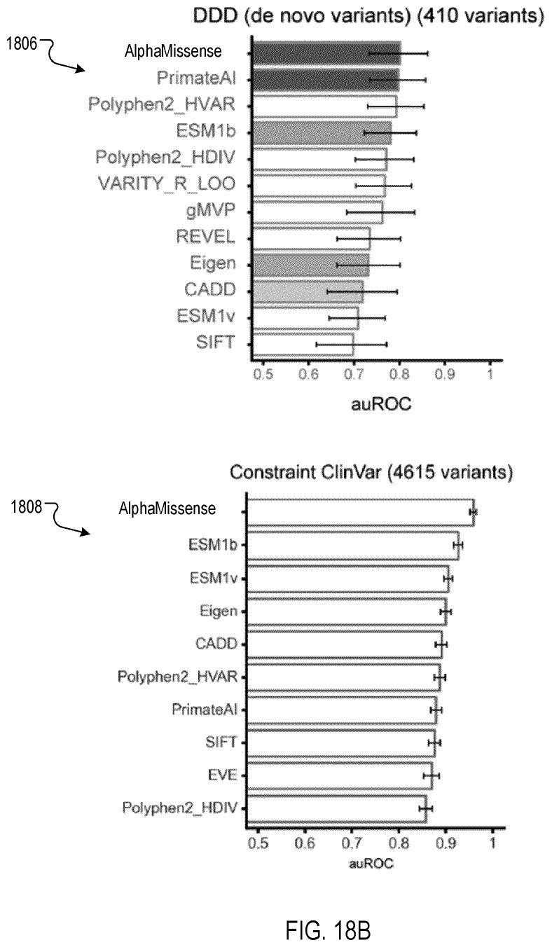

FIG. 18A and FIG. 18B illustrate the performance (e.g., classification accuracy) of the system described in this specification on clinically curated classification benchmarks evaluated by area under the receiver operator curve (auROC). Error bars show the 95% confidence interval of 1000 bootstrap resamples. Chart 1802 compares the performance of the present system (“AlphaMissense”) and other predictors on held-out 82,868 ClinVar missense variants. Chart 1804 compares the performance of the present system (“AlphaMissense”) and other predictors on 868 cancer mutations in hotspots from 202 driver genes versus 1734 randomly selected negative variants. Chart 1806 compares the performance of the present system (“AlphaMissense”) and other predictors on distinguishing de novo variants from Deciphering Developmental Disorders cohort (DDD) patients and health controls; 353 patient variants and 57 control variants from 215 DDD related genes are considered. Chart 1808 compares the performance of the present system (“AlphaMissense”) and other predictors on classification of ClinVar (3430 pathogenic and 1185 benign) variants on regions of high evolutionary constraint.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Certain novel aspects of the subject matter of this specification are set forth in the claims below, accompanied by further description in Appendix A.

What is claimed is:

1. A method performed by one or more computers, the method comprising:
generating a pathogenicity score characterizing a likelihood that a mutation to a protein is a pathogenic mutation,
wherein the mutation modifies an amino acid sequence of the protein by replacing an original amino acid by a substitute amino acid at a mutation position in the amino acid sequence of the protein,
wherein generating the pathogenicity score comprises:
generating a network input to a pathogenicity prediction neural network, wherein the network input comprises a multiple sequence alignment (MSA) representation that represents an MSA for the protein;
processing the network input using the pathogenicity prediction neural network to generate a score distribution over a set of amino acids; and
generating the pathogenicity score based on a difference between: (i) a score, under the score distribution, for the original amino acid, and (ii) a score, under the score distribution, for the substitute amino acid.

2. The method of claim 1, wherein the MSA representation comprises a respective embedding corresponding to each position in an amino acid sequence of each protein in the MSA; and
wherein generating the MSA representation comprises:
masking an embedding in the MSA representation that corresponds to the mutation position in the amino acid sequence of the protein. The method of claim 2, wherein processing the network input using the pathogenicity prediction neural network to generate the score distribution over the set of amino acids comprises:
processing the MSA representation using an embedding subnetwork of the pathogenicity prediction neural network to generate an updated MSA representation, wherein the updated MSA representation comprises a respective updated embedding corresponding to each position in the amino acid sequence of each protein in the MSA; and
processing the updated embedding corresponding to the mutation position in the amino acid sequence of the protein using a projection subnetwork of the pathogenicity prediction neural network to generate the score distribution over the set of amino acids.

3. The method of claim 2, wherein the embedding subnetwork of the pathogenicity prediction neural network comprises one or more self-attention neural network layers.

4. The method of claim 3, wherein the embedding subnetwork of the pathogenicity prediction neural network comprises one or more row-wise or column-wise self-attention neural network layers.

5. The method of claim 1, wherein the pathogenicity prediction neural network has been trained by operations comprising:
training the pathogenicity prediction neural network to perform a protein unmasking task; and
training the pathogenicity prediction neural network to perform a pathogenicity prediction task.

6. The method of claim 5, wherein training the pathogenicity prediction neural network to perform the protein unmasking task comprises, for each of a plurality of training proteins:
generating a network input to the pathogenicity prediction neural network based on the training protein, wherein:
the network input comprises a MSA representation representing an MSA for the training protein; and
the MSA representation comprises a respective masked embedding corresponding to each of one or more masked positions in an amino acid sequence of the training protein;
processing the network input to the pathogenicity prediction neural network to generate a network output that, for each masked position in the amino acid sequence of the training protein, defines a respective prediction for an identity of an amino acid located at the masked position in the amino acid sequence of the training protein; and
backpropagating gradients of a masking loss through the pathogenicity prediction neural network, wherein the masking loss measures an accuracy of the respective prediction generated by the pathogenicity prediction neural network for each masked position.

7. The method of claim 5, further comprising:
training the pathogenicity prediction neural network to perform a protein structure prediction task.

8. The method of claim 7, wherein training the pathogenicity prediction neural network to perform the protein structure prediction task comprises, for each of a plurality of training proteins:
generating a network input to the pathogenicity prediction neural network based on the training protein, wherein the network input comprises a MSA representation that represents a MSA for the training protein;
processing the network input using the pathogenicity prediction neural network to generate an intermediate output of pathogenicity prediction neural network;
processing the intermediate output of the pathogenicity prediction neural network using a folding neural network to generate a set of structure parameters defining a predicted structure of the training protein; and
backpropagating gradients of a structure loss through the folding neural network and into the pathogenicity prediction neural network, wherein the structure loss measures an error in the predicted structure of the training protein.

9. The method of claim 5, wherein the pathogenicity prediction neural network is pre-trained to perform the protein unmasking task and the protein structure prediction task prior to being trained to perform the pathogenicity prediction task.

10. The method of claim 1, wherein the pathogenicity prediction neural network has been trained over at least two stages of training comprising a first training stage and a second training stage;
wherein the first training stage comprises:
training the pathogenicity prediction neural network on a set of pathogenicity training examples, wherein each pathogenicity training example defines: (i) an amino acid sequence of a training protein, (ii) one or more mutations to the amino acid sequence of the training protein, and (iii) a respective target pathogenicity score for each of the one or more mutations;
determining a respective filtering score for each training example; and
filtering the set of pathogenicity training examples using the filtering scores.

11. The method of claim 10, wherein for each training example, determining the filtering score for the training example comprises:
determining, for each mutation included in the training example, a predicted pathogenicity score for the mutation using the pathogenicity prediction neural network; and
determining, for each mutation included in the training example, an error score for the mutation based on an error between: (i) the predicted pathogenicity score for the mutation, and (ii) the target pathogenicity score for the mutation; and
determining the filtering score for the training example based at least in part on the error scores for the mutations included in the training example.

12. The method of claim 10, wherein filtering the set of pathogenicity training examples using the filtering scores comprises:
determining a probability distribution over the set of pathogenicity training examples based on the filtering scores;
sampling a subset of the pathogenicity training examples from the set of pathogenicity training examples in accordance with the probability distribution; and
removing the sampled subset of pathogenicity training examples from the set of pathogenicity training examples.

13. The method of claim 10, wherein the second training stage comprises:
re-training the pathogenicity prediction neural network on the filtered set of pathogenicity training examples.

14. The method of claim 1, further comprising:
determining that the pathogenicity score for the mutation satisfies a threshold; and
in response to determining that the pathogenicity score for the mutation satisfies the threshold:
classifying the mutation as being a pathogenic mutation.

15. The method of claim 14, wherein determining that the pathogenicity score for the mutation satisfies the threshold comprises:
determining that the pathogenicity score for the mutation exceeds the threshold.

16. The method of claim 1, further comprising using the pathogenicity score to identify a cause of a disease in a subject.

17. The method of claim 1, used for obtaining a mutated protein for pest control, the method further comprising:
determining the pathogenicity score for a plurality of mutations of a reference protein each mutation defining a respective mutated protein; and
selecting one of the mutations to define the mutated protein for pest control, based on the determined pathogenicity scores.

18. The method of claim 1, used for screening one or more living organisms for the presence of a protein that has a pathogenic mutation, the method further comprising:
obtaining the amino acid sequence of the protein for each of the organisms;
for each of the obtained amino acid sequences that includes a mutation, determining the pathogenicity score for the mutation; and
processing each determined pathogenicity score to determine whether the mutation is pathogenic.

19. (canceled)

20. (canceled)

21. A system comprising:
one or more computers; and
one or more storage devices communicatively coupled to the one or more computers, wherein the one or more storage devices store instructions that, when executed by the one or more computers, cause the one or more computers to perform operations comprising:
generating a pathogenicity score characterizing a likelihood that a mutation to a protein is a pathogenic mutation,
wherein the mutation modifies an amino acid sequence of the protein by replacing an original amino acid by a substitute amino acid at a mutation position in the amino acid sequence of the protein,
wherein generating the pathogenicity score comprises:
generating a network input to a pathogenicity prediction neural network, wherein the network input comprises a multiple sequence alignment (MSA) representation that represents an MSA for the protein;
processing the network input using the pathogenicity prediction neural network to generate a score distribution over a set of amino acids; and
generating the pathogenicity score based on a difference between: (i) a score, under the score distribution, for the original amino acid, and (ii) a score, under the score distribution, for the substitute amino acid.

22. One or more non-transitory computer storage media storing instructions that when executed by one or more computers cause the one or more computers to perform operations comprising:
generating a pathogenicity score characterizing a likelihood that a mutation to a protein is a pathogenic mutation,
wherein the mutation modifies an amino acid sequence of the protein by replacing an original amino acid by a substitute amino acid at a mutation position in the amino acid sequence of the protein,
wherein generating the pathogenicity score comprises:
generating a network input to a pathogenicity prediction neural network, wherein the network input comprises a multiple sequence alignment (MSA) representation that represents an MSA for the protein;
processing the network input using the pathogenicity prediction neural network to generate a score distribution over a set of amino acids; and
generating the pathogenicity score based on a difference between: (i) a score, under the score distribution, for the original amino acid, and (ii) a score, under the score distribution, for the substitute amino acid.

23. (canceled)

24. (canceled)

25. (canceled)

26. (canceled)

27. (canceled)
